(19) United States
(12) Patent Application Publication
(10) Pub. No.: US 2024/0118266 A1
(43) Pub. Date: Apr. 11, 2024

(54) CELL DEATH BIOMARKER

(71) Applicant: UCL Business PLC, London (GB)

(72) Inventors: Samuel Janes, Harpenden (GB); Krishna Kolluri, London (GB); Ultan McDermott, Hinxton (GB); Neelam Kumar, London (GB)

(21) Appl. No.: 18/299,415

(22) Filed: Apr. 12, 2023

Related U.S. Application Data
(62) Division of application No. 16/333,590, filed on Mar. 14, 2019, now Pat. No. 11,789,012.

(30) Foreign Application Priority Data
Sep. 16, 2016 (GB) 1615842.0

(51) Int. Cl. G01N 33/50; A61K 31/7105; A61P 35/00; G01N 33/574

(52) U.S. Cl. CPC G01N 33/5017; A61K 31/7105; A61P 35/00; G01N 33/5023; G01N 33/574; G01N 2496/05; G01N 2500/02; G01N 2500/10; G01N 2510/00; G01N 2800/52

(57) ABSTRACT

The invention relates to cell death of cancer cells, and in particular to biomarkers that may be used to identify cancer cells that are sensitive to death receptor ligand (DRL)-induced cell death. The invention also extends to prognostic methods and kits for identifying cancer cells that are sensitive to DRL-induced cell death. The invention further extends to novel compositions and therapeutic methods using such compositions for treating cancer.

### CROSS-REFERENCE TO RELATED APPLICATIONS

This application is a divisional application that claims the benefit of U.S. patent application Ser. No. 16/333,590, filed Mar. 14, 2019, which is the National Stage of International Application No. PCT/GB2017/052733 (International Publication No. WO2018/051,110), filed Sep. 15, 2017, which claims priority to UK Patent Application Serial No. 1615842.0, filed Sep. 16, 2016, the contents of which are incorporated herein by reference.

The present invention relates to cell death of cancer cells, and in particular to biomarkers that may be used to identify cancer cells that are sensitive to death receptor ligand (DRL)-induced cell death. The invention also extends to prognostic methods and kits for identifying cancer cells that are sensitive to DRL-induced cell death. The invention further extends to novel compositions and therapeutic methods using such compositions for treating cancer.

Malignant pleural mesothelioma (MPM) is a rare but invariably fatal malignancy that occurs most frequently in the pleura and is almost always associated with asbestos exposure (1, 2). Although asbestos mining and usage has been the subject of legislation in many nations, there remains evidence of increased use and production of asbestos as the trend towards global industrialisation increases (3). Given a cancer latency period of anywhere from 10-40 years, it is therefore likely that in such countries asbestos-related cancers will increase over the next five decades. Current treatment options for patients diagnosed with MPM are limited. Radical surgery, for example, offers limited benefit and the cancer is refractory to most chemotherapeutic agents (4). Indeed, the current gold standard chemotherapy regimen of pemetrexed plus cisplatin offers a median overall survival of 12.1 months and a time to progression of 5.7 months (5).

Tumour necrosis factor (TNF)-related apoptosis-inducing ligand (TRAIL/Apo2L) is a member of the TNF ligand family that induces cell death in tumour cells in vitro and in vivo but not in most normal cells. Numerous chemotherapeutic drugs have also been shown to sensitize tumour cells to TRAIL-mediated cell death. While some mesothelioma cells are sensitive to TRAIL induced cell death others are resistant to the induction of cell death in vitro and this disruption of core-cell death machinery has been implicated in the general resistance to conventional cytotoxic agents observed clinically (11).

Certain cancers are sensitive to the pro-cell death effects of known death receptor ligands, while others are not. Without knowledge of which cancers are sensitive to DRL-induced cell death, individuals with cancer must rely on treatment with non-specific cytotoxic therapies, such as chemotherapy, with the toxicities and problems described (5). Furthermore, with no clear indication pre-treatment of whether a patient will respond or not, the benefit of such treatment is uncertain at the outset.

There is thus a compelling need for more effective therapeutic interventions for the treatment of cancers that are sensitive to cell death caused by death receptor ligand (DRL)-induced cell death. There is also a need to provide a biomarker that can be used to identify cancerous cells that are sensitive to cell death caused by death receptor ligands when bound to death receptors, such as TRAIL-receptor 1 & 2 (Death Receptors 4&5), TNF receptor and FAS receptor

Hence, in a first aspect of the invention, there is provided a method of determining if an individual's cancer cell is sensitive to death receptor ligand (DRL)-induced cell death, the method comprising detecting, in a biological sample taken from the individual, for:
(i) the presence of a mutant BAP1 gene or mutant BAP1 protein;
(ii) a reduced level of expression of a wild-type BAP1 gene or a lower wild-type BAP1 protein concentration compared to the level of expression or protein concentration in a reference cell that is a BAP1 wildtype cell, which is resistant to DRL-induced cell death; or
(iii) reduced or non-binding of an ASXL protein to a wild-type BAP1 protein compared to the level of binding in a reference cell that is a BAP1 wild-type cell, which is resistant to DRL-induced cell death;
   wherein the presence, in the sample, of the mutant BAP1 gene or the mutant BAP1 protein, or of a reduced level expression of the wild-type BAP1 gene or a lower protein concentration, or reduced or non-binding of an ASXL protein to a wild-type BAP1 protein, is indicative of the individual's cancer cell being sensitive to DRL-induced cell death.

Previous genetic analyses have identified several key recurrent alterations, including inactivation of CDKN2A, NF2 and BAP1, as well as frequent losses and gains of a number of chromosomal arms. More recently, the first whole exome sequence analysis of 22 malignant mesothelioma patients confirmed the importance of these three genes as probable driver events in tumour development as well as identifying recurrent non-synonymous mutations in CULL, an essential component of the SCF E3 ubiquitin ligase complex (6).

BAP1 is a tumour suppressor gene that is somatically mutated in a variety of cancer types including mesothelioma (7), uveal melanoma (8), renal cell carcinoma (9) and cholangiocarcinomas (10). The inventors have discovered that the expression of BAP1 mutants in cancer cells causes increased sensitivity to cell death induced by death receptor ligands, which exhibit fewer off-target effects. Tumour necrosis factor (TNF)-related apoptosis-inducing ligand (TRAIL), for example, induces cell death in tumour cells in vitro and in vivo but not in most normal cells. Advantageously, the invention therefore increases the likelihood that an individual with a cancer, which is associated with expression of a mutant BAP1 gene or synthesis of a mutant BAP1 protein, or a reduced level of expression of a wild-type BAP1 gene or a lower wild-type BAP1 protein concentration, will exhibit a positive outcome when treated with death receptor ligand therapy. Accordingly, the method of the invention provides a prognosis. Thus, such individuals are less likely to be treated with non-specific therapies and suffer the associated undesirable side-effects.

BAP1 is a deubiquitinating enzyme encoded by the BAP1 gene. It is an 80.4 kDa protein, which comprises a nuclear-localising sequence and a ubiquitin carboxy-terminal hydrolase (UCH) domain, which provides BAP1 with deubiquitinase activity. The BAP1 gene may be mutated in the germline or somatically in a variety of forms of cancer. The nucleotide sequence encoding one embodiment of the human wild-type BAP1 gene (BAP1 Whole gene sequence >gi|568815595:c52410105-52401004 Homo sapiens chromosome 3) is provided herein as SEQ ID No.1, as follows:

[SEQ ID No. 1]
GAGCGCATGCCCGCATCTGCTGTCCGACAGGCGGAAGACGAGCCCAGAGG
CGGAGCAGGGCCGTCGCGCCTTGGTGACGTCTGCCGCCGGCGCGGGCGGG
TGACGCGACTGGGCCCGTTGTCTGTGTGTGGGACTGAGGGGCCCCGGGGG
CGGTGGGGGCTCCCGGTGGGGGCAGCGGTGGGGAGGGAGGGCCTGGACAT
GGCGCTGAGGGGCCGCCCCGCGGGAAGATGAATAAGGGCTGGCTGGAGCT
GGAGAGCGACCCAGGTGAGGAGGGGACCGGGAGGGCCAGGGGCTGGGGAG
GCCGGATGGGCCCGGGACGCGCCTGCCTGACCATCACCCCCTCCTCTTGT
CGCCCCACCCAGGCCTCTTCACCCTGCTCGTGGAAGATTTCGGTAAGAGC
CTTTTCTCCCTGCCGGACCGGGGCTGTGGCGGCCCACCCCTGCGCCCTCA
CTCATCAGGGGCTGTCCTTCCCTACTGCTTTCCTTTCCTCATCGCAGGTG
TCAAGGGGGTGCAAGTGGAGGAGATCTACGACCTTCAGAGCAAATGTCAG
GGGTGAGTGGCTGTACACCAGGGCTGCCCCTTACACCCAGAGTGCTGGGG
AAGGTCCCAGAGAACAGGGCCCCTTAGGGAAGACAGTGCCAGGAACCCTA
CGTTGTAAAATCTCACAGAAAGCAGCAGCCTTGCTCTCTGAGTGCCCGCT
CCTGATCAAACTGATACTTTCTTTTCTCCCAAACTTTCCTTAGCGCTTCC
CTTTTTGTAGCAGCCCCCTCCCCACCCCTAAGCATCCTTTGGTTCAGCTG
CTTTCCTGGCCTTGCAGCGGGAAGACCCCGGTCACACAATGTCTTTTGTG
CAGTTGTGTAATGTATTAATTTTAGTGTGCCCATGTGTCCTTGGCTTTAA
TCCTGACACAAAGTCATCCTGTATTGATTGGTTGGGGTGACAAGGCCCCT
CCTGGGTGCCCACACTTAGAGTCTTTTCCCAGTGGTCCTGCAGAATAGAT
GTGTAAGAGAGTAGCAACAGTAGCAACCGTGACTGAACCAAGAAGTCTAC
TTTAATTTCCTGGAACAAAAGAGACTGGTGTGGGTGTTCATTTGCTTTCC
TGACTGCATTGGGGCCCACAAGTGAGAAGGAGTGCCTCAGTTCCTCATCA
GAGTTTTTGTTCTTGTCTTACTTTGTGTTCCTACCCTGTCCCATCCTTGG
CCCTCAGTTCCAGCTTTTCTTCTCTTACCCAGAACTATAGACTTCATAAG
GAGACTGGGTGGACTCCTGGAGCATCACAGTCAGAGGCTTATGCTTTGCT
CTGCCTGTGGCAGGCCTTTGGTGTGTGAGGGCACAAGGCCACTTCAGACA
CAGTGTTGGGAAGAAGCCAGGGGAGAGGGGGGATCACAGCAAGGACACCT
GAGTGATGACGCAGTGCAAAGGATTAATGGGAGAAAGAAGGGAATGCTGA
TTGTCTTCTCCCCTTTGGCTGATCTGGCTCTGCCCCTTACTTCCCCCAGC
CCTGTATATGGATTTATCTTCCTGTTCAAATGGATCGAAGAGCGCCGGTC
CCGGCGAAAGGTCTCTACCTTGGTGGATGATACGTCCGTGATTGATGATG
ATATTGTGAATAACATGTTCTTTGCCCACCAGGTCTGCTGGACTCTGTGC
TTTGTTTGGAGGGTGGGATGCTGCCATGTTTTTGCTTGGGAAGTGGAAAT
GGAGGAAGACAGGAGGAGGAGATAGGCAGATTCTAGGGGTGGTAGCTACA
GAAATCCTCTGGCAGAACGAACTGAACTCTTAATTCATTAAAGGGAACAG
CTTTAGAGTAGGAGGGTGTCTGAGTCCACTCTCTGTGTCCTCAGATATCC
AGTGGGTATTTGGTAGGTGCTTGTTAAATGAATAAACATTAGGCAAAGAT
GAAAGGAGCTGAGAAGGGGAGTTGTCCAGATATGACTGACCTGCTCTGGA
TCCCCATTCTTGATGTATATGGGCTTGGGGCTTGCAGTGAGGGGTGCTGT
GTATGGGTGACTATTCTTGGTTTCACAGCTGATACCCAACTCTTGTGCAA
CTCATGCCTTGCTGAGCGTGCTCCTGAACTGCAGCAGCGTGGACCTGGGA
CCCACCCTGAGTCGCATGAAGGACTTCACCAAGGGTTTCAGCCCTGAGGT
AGGCTGCAGTGCCTTCATCCTGGCTCACAGCCAACTGGGCAGATCTGACC
CTGAGGGCCACTGGGAATGCTACCACATGATATTGGGTACTATTAGGCTG
TTTCTTTTTCAAATGATTGTTTATGTTACATTTGACTCTTAAATAAATTG
TGTAAGGCCATTGTTTTTAGATGCAGTTGCGGGGAAAGGACACAGGCCTA
GGGAGGGAGGAGAGTTTCCTTAAGTCAGACCATGTCAGAACCTTCTCTGT
CAGGACTTTTCCTCTCAGGCCATGTTGCTTCCTAGTGTCCACTAATTACC
ATGCAAGGCCAGCACAGTCCATCTCTTTGGGGCTCCAGAGCTCTTTTCTG
CCCCCACCAGCCTTTTAAGAAAGTTCGTCTGTGTTCCTTCCGATTCCTGG
AATGCCTCCAGGCTGCTCTCTGAAGCTTTGCCTTCCACCCATAGTCCTAC
CTGAGGAGAAATTATTCTGATACGGCCTTATTTTCTTCCCCGTAGAGCAA
AGGATATGCGATTGGCAATGCCCCGGAGTTGGCCAAGGCCCATAATAGCC
ATGCCAGGTGTGTGGGAGCTGTGGGAGCTGATGTGGGGTGGGAGTAGGGG
GAGTATCATTTTTTGGGCCCTGACTCTGTTTTTCCCCAGGCCCGAGCCAC
GCCACCTCCCTGAGAAGCAGAATGGCCTTAGTGCAGTGCGGACCATGGAG
GCGTTCCACTTTGTCAGCTATGTGCCTATCACAGGCCGGCTCTTTGAGCT
GGATGGGCTGAAGGTCTACCCCATTGACCATGGTAGGCACCATGAGCTGG
AGGCCTGTTGGGTGTCTCTGCCTACCTCCTAGGGAGCTGGGGCTCAGGGC
CCTCTGGTATGTGGTACCCAGTGGCAGGGGTTGTCGGTACCGACACCCGG
CTCTGGCTGGGGTTTCACCCTACACCATATTGCCCGACCAGCTCCTGATT
CCCTGGCTCAACTGCTCTTCTCTGTCTTCCTTCCCACTCCTGGCCTGCCC
AAACTCAGGGTTTCCTTCTCGCTGATTCCTTGTCTTGGTCTCCACTAGGG
CCCTGGGGGGAGGACGAGGAGTGGACAGACAAGGCCCGGCGGGTCATCAT
GGAGCGTATCGGCCTCGCCACTGCAGGGTAAGGGCCCTGTGCCTGCCCTG
TTCTACTCTCTGGAGCTGTACCTACTTTGGGAGGGACAGAGAGTATCCAG
GTGATTTGTAAATTGCAAGGCCATATGGTGAATCTGGCAAGATCAGGCTT
AGATCATGGGTTCTCAACTTGTTGTCTTATTTCCTGCCTGGGCTGCCTGT
GGCCTGCTCCTGGGTGGGCTGGGGGAGGGGCAGGCCTCAGTGGAGCCTTA
GGCAGCCCAGGTCTGCTGGTTCACTTCCAGATAGGCCCCTCATACAGCTT
GTTGGAAGGTACCAGCTCAGGTGCCTGGCATGTATGGCTAGTCGCTGCCT
GCCTGTTGGGGTGGGGCCTATACCTACAGCTGCAGGTGTGACTGCAGGGA
GCCCTGCCAGGATATCTGCCTCAACCTGATGGCGGGGCCGGGGCGGGAGC
TGCTCTCACGGCTGCGGCTGTGACTGCAGGGAGCCCTACCACGACATCCG
CTTCAACCTGATGGCAGTGGTGCCCGACCGCAGGATCAAGTATGAGGCCA
GGCTGCATGTGCTGAAGGTGAACCGTCAGACAGTACTAGAGGCTCTGCAG
CAGGTAGGTGCCCTTTCTTCCTGGCCTCTGCCCAGCCCAACCCTCCCTGC
ATTCCTCCTCCCTTCCCCCACAGCATTTGTCTCTGATTCGTGAACATACT
CTCTTGTAGATCTGGGCTTCAGCTAACCACATCTTTTCTTTGCCCCCATT
GTGGGAAAGGTGGGACTTGGAGTGGGGAGGGAGAATAGCTTCTAAAAGGA
AGTTTGGGTTTGGGTGTTTTATTTCCCTGTGAGTGAATGGGTAGAGCCAA
GGCCATTATTCCTTTAGGTCCTCAGCCCTTAGCTATTTAAGGTAGAAGCC
CGGGTCTACCCTTTCTCCTCTGAGCCCTGGATTCTGTTGTTAGCTGATAA
GAGTAACACAGCCAGAGCTGATTCAGACCCACAAGTCTCAAGAGTCACAG
CTGCCTGAGGAGTCCAAGTCAGCCAGCAACAAGTCCCCGCTGGTGCTGGA
AGCAAACAGGGCCCCTGCAGCCTCTGAGGGCAACCACACAGGTACTGGGG
GGTTTGGGACCTCTTGTGGACCTCAGAGCCACCCGCTAATGTCTGACATG
GGAGGCCTAAACAGGGAAAGTCTTTTTCTGGGGATGTCCTTGGGCAGTGT
TCTTCCCCCGTCAGAAGGTAGAGGGAGAGCAGTCCTTCCCTAAAGAAAGG
CACCTGTAAAGGGCCGCTGTTACCACAGGCCCCTGGGCCCTTCTCTGTAA
TGTACACTCCCTTTCTTGTTTTCTCTAGAGGCGGTTTTTTTTTTTTTTTT
TTTTTTTTTTTTTCTTCCTGCTTCTTTTTTCCCATCTCATTCTTTGCCCT
GTCTCATTGCGGGATCATGACTTAGAGCTTGCTGACTCCCATTGCACCAG
CTGGCTGGGCTGTTCTTCTCTGGGAAGTGCTGGTTCACAGGGCCGGGGAG
ACTGTGAGCTTTTCTTGGAGATCCTACTGGAGGTCCTGCCTGTGTTCTTG
CCCTGTCTCAGATGGTGCAGAGGAGGCGGCTGGTTCATGCGCACAAGCCC
CATCCCACAGCCCTCCCAACAAACCCAAGCTAGTGGTGAAGCCTCCAGGC
AGCAGCCTCAATGGGGTTCACCCCAACCCCACTCCCATTGTCCAGCGGCT
GCCGGCCTTTCTAGACAATCACAATTATGCCAAGTCCCCCATGCAGGTAA
GCTGGGAGCACCCTTGCAGGATTCTCTACTTGATTCTCTTGAGAGGCTGC
AACAGGCAATTTTCCCATGTGGTTCCTTGGTGTTCATCCTTGGCATGGCT
GGGTCAAGCTGCCTGGGCCTGGGTTGCTAGGTTCCTCTGCCTGATATGAA
AAGGCCCCCACAACAGCAGGAGCTTAGGGAGGCAGGGAGAGCTCCTTTGA
ATTTAATCTAGTTACGTGGCTGTGGGATTAAATGTTTAGGTCACGCTCCT
TGGTACAACTTCATGGGTTGGGTTTTACTGGCAAAATAAAGGCATGTGTT
TCAGGGCACTCTGTTTCTCTTAAAACCCCTCCGTGGGGTTCTATCCAGTG
TAAGTGGGTGGCAGCCTCCCCACAAGCCAAGGACAGGCCATGGAACAGCT
GGAGGGGTTCCGCTGACTCAGTCTGGAAAACCATGTTGGCTTTCTCTCTG
GCTGTGAGTGTCTAGGCTCAGCCTGGGCCGAGCAGCACTTGTTTGTAACT
GCCCTGGTCTTTGTCCCAGGAGGAAGAAGACCTGGCGGCAGGTGTGGGCC
GCAGCCGAGTTCCAGTCCGCCCACCCCAGCAGTACTCAGATGATGAGGAT
GACTATGAGGATGACGAGGAGGATGACGTGCAGAACACCAACTCTGCCCT
TAGGTCAGCCCAGCTTTCTAAGGCTACCAGGTTCTAGGTGCTTCGGATCC
CATCCTGAATATCTCAGTCTGTGTCTGAGAATGCCCTGCAGCAGATAATG
TTGAGCACCTGCGGAGTTTGGGGCCCTGGGGGAGGCTGGCATGATGGGGC
TGACCCCAGGTCCCCAGGAAGTTTTTGGTGGGCTGGGGGGTAAGGCTGAG
CACGTAAGCTTATATCATGTCCTATTGGAAGTGGCCTTTTAGCCAGGCCT
TGAAGGATTGGTTGGGGCAGGGATGGAGGAGATGTGGGTGGTGGGGAGGC
AGCTTTGCTGGAACACAGGGCATTGGCAAAAGGCCAGGAGTGGGATGGCT
GGAATAGAGGAAGTGTCTTTTGAGGACACTTGGCTGCAGCTGTCAGAACT
TGATGCCAGGCTTAGCATGGCTAGTTCAAGTTGCTTGGACCAAGTATAAG
GAGTTTTAGGGTCAGCCCCTGGAGGTCGGGATGTATTTAAGCCATTCTGG
GTACTGCTGGGTATGGTCACCTGGCCCGTTCCCTTGCTTCACATCTTCTC
GGGCCCCACAGGTATAAGGGGAAGGGAACAGGGAAGCCAGGGGCATTGAG
CGGTTCTGCTGATGGGCAACTGTCAGTGCTGCAGCCCAACACCATCAACG
TCTTGGCTGAGAAGCTCAAAGAGTCCCAGAAGGACCTCTCAATTCCTCTG
TCCATCAAGACTAGCAGCGGGGCTGGGAGTCCGGCTGTGGCAGTGCCCAC
ACACTCGCAGCCCTCACCCACCCCCAGCAATGAGAGTACAGACACGGCCT
CTGAGATCGGCAGTGCTTTCAACTCGCCACTGCGCTCGCCTATCCGCTCA
GCCAACCCGACGCGGCCCTCCAGCCCTGTCACCTCCCACATCTCCAAGGT
GCTTTTTGGAGAGGATGACAGCCTGCTGCGTGTTGACTGCATACGCTACA
ACCGTGCTGTCCGTGATCTGGGTCCTGTCATCAGCACAGGCCTGCTGCAC
CTGGCTGAGGATGGGGTGCTGAGTCCCCTGGCGCTGACAGGTGGGCCTTG
GACTGGCTCACTGGCCACTTGGTGCACCCAGGAGGGAGGAGGGAAGTGGC
CAAGTGACCACAAAGTGTCCTGCACTCTGATGATTTTCTTGTGACCTCTC
TTCCCAGAGGGTGGGAAGGGTTCCTCGCCCTCCATCAGACCAATCCAAGG
CAGCCAGGGGTCCAGCAGCCCAGTGGAGAAGGAGGTCGTGGAAGCCACGG
ACAGCAGAGAGAAGACGGGGATGGTGAGGCCTGGCGAGCCCTTGAGTGGG
GAGAAATACTCACCCAAGGTGAGCCTCCGTTGTGGTTTTCTCCTTTAATC
CTGGCAGAGGGTAAGGCCTGAGCTCCTCCTGCCCAGGTGCCAAGTTCTTG
ATTGGAACTTTGGTGTGAAGATTGGTGGCTGGAGCCATGTGCCAGAAGAC
TTTCTGGGTTGGGTGGTGGCAGGGGCCTTGATAGGCATGGACTCGCTGCT
CATCCTTGCCTCTAGCTGCCTATTGCTCGTGGGGCTTTGTTGCTGGCCCG
CCCCGATCAGAGGTGCAATGCTGGGTTTTGGCAGGAGCTGCTGGCACTGC
TGAAGTGTGTGGAGGCTGAGATTGCAAACTATGAGGCGTGCCTCAAGGAG
GAGGTAGAGAAGAGGAAGAAGTTCAAGGTGGGTGATTTCTCCAGTTGCCT
GATCTGGCCTCTCCCGAGGTCCACTGGTGGCTGCTCTGGCAAGATTGGCT
CCAGTGCTCTCAGTCTTCTTCTCTCCTACAGATTGATGACCAGAGAAGGA
CCCACAACTACGATGAGTTCATCTGCACCTTTATCTCCATGCTGGCTCAG
GAAGGTGAGGGGATGCGCTGCTGTCTTAACTGGAATGCCCTGCTGAGGGC
CGTGTCCTTCAGCTCCCCTCCCCTGGCCTCTCCTGAGGCTTGAGCAGACC
TTGGGGCACAGGGAGGGCCATGAGAGCCTCAGCTCCTGGCCTGAGGCAGC
CAGCACCTGCTCAAGGGTCTCTACCTCTTCGCAGGCATGCTGGCCAACCT
AGTGGAGCAGAACATCTCCGTGCGGCGGCGCCAAGGGGTCAGCATCGGCC
GGCTCCACAAGCAGCGGAAGCCTGACCGGCGGAAACGCTCTCGCCCCTAC
AAGGCCAAGCGCCAGTGAGGACTGCTGGCCCTGACTCTGCAGCCCACTCT
TGCCGTGTGGCCCTCACCAGGGTCCTTCCCTGCCCCACTTCCCCTTTTCC
CAGTATTACTGAATAGTCCCAGCTGGAGAGTCCAGGCCCTGGGAATGGGA
GGAACCAGGCCACATTCCTTCCATCGTGCCCTGAGGCCTGACACGGCAGA
TCAGCCCCATAGTGCTCAGGAGGCAGCATCTGGAGTTGGGGCACAGCGAG
GTACTGCAGCTTCCTCCACAGCCGGCTGTGGAGCAGCAGGACCTGGCCCT
TCTGCCTGGGCAGCAGAATATATATTTTACCTATCAGAGACATCTATTTT
TCTGGGCTCCAACCCAACATGCCACCATGTTGACATAAGTTCCTACCTGA
CTATGCTTTCTCTCCTAGGAGCTGTCCTGGTGGGCCCAGGTCCTTGTATC
ATGCCACGGTCCCAACTACAGGGTCCTAGCTGGGGGCCTGGGTGGGCCCT
GGGCTCTGGGCCCTGCTGCTCTAGCCCCAGCCACCAGCCTGTCCCTGTTG
TAAGGAAGCCAGGTCTTCTCTCTTCATTCCTCTTAGGAGAGTGCCAAACT
CAGGGACCCAGCACTGGGCTGGGTTGGGAGTAGGGTGTCCCAGTGGGGTT
GGGGTGAGCAGGCTGCTGGGATCCCATGGCCTGAGCAGAGCATGTGGGAA
CTGTTCAGTGGCCTGTGAACTGTCTTCCTTGTTCTAGCCAGGCTGTTCAA
GACTGCTCTCCATAGCAAGGTTCTAGGGCTCTTCGCCTTCAGTGTTGTGG
CCCTAGCTATGGGCCTAAATTGGGCTCTAGGTCTCTGTCCCTGGCGCTTG
AGGCTCAGAAGAGCCTCTGTCCAGCCCCTCAGTATTACCATGTCTCCCTC
TCAGGGGTAGCAGAGACAGGGTTGCTTATAGGAAGCTGGCACCACTCAGC
TCTTCCTGCTACTCCAGTTTCCTCAGCCTCTGCAAGGCACTCAGGGTGGG
GGACAGCAGGATCAAGACAACCCGTTGGAGCCCCTGTGTTCCAGAGGACC
TGATGCCAAGGGGTAATGGGCCCAGCAGTGCCTCTGGAGCCCAGGCCCCA
ACACAGCCCCATGGCCTCTGCCAGATGGCTTTGAAAAAGGTGATCCAAGC
AGGCCCCTTTATCTGTACATAGTGACTGAGTGGGGGGTGCTGGCAAGTGT
GGCAGCTGCCTCTGGGCTGAGCACAGCTTGACCCCTCTAGCCCCTGTAAA
TACTGGATCAATGAATGAATAAAACTCTCCTAAGAATCTCCTGAGAAATG
AA

The cDNA sequence encoding one embodiment of the human wild-type BAP1 gene is provided herein as SEQ ID No. 2, as follows:

[SEQ ID No. 2]
ATGAATAAGGGCTGGCTGGAGCTGGAGAGCGACCCAGGCCTCTTCACCCT
GCTCGTGGAAGATTTCGGTGTCAAGGGGGTGCAAGTGGAGGAGATCTACG
ACCTTCAGAGCAAATGTCAGGGCCCTGTATATGGATTTATCTTCCTGTTC
AAATGGATCGAAGAGCGCCGGTCCCGGCGAAAGGTCTCTACCTTGGTGGA
TGATACGTCCGTGATTGATGATGATATTGTGAATAACATGTTCTTTGCCC
ACCAGCTGATACCCAACTCTTGTGCAACTCATGCCTTGCTGAGCGTGCTC
CTGAACTGCAGCAGCGTGGACCTGGGACCCACCCTGAGTCGCATGAAGGA
CTTCACCAAGGGTTTCAGCCCTGAGAGCAAAGGATATGCGATTGGCAATG
CCCCGGAGTTGGCCAAGGCCCATAATAGCCATGCCAGGCCCGAGCCACGC
CACCTCCCTGAGAAGCAGAATGGCCTTAGTGCAGTGCGGACCATGGAGGC
GTTCCACTTTGTCAGCTATGTGCCTATCACAGGCCGGCTCTTTGAGCTGG
ATGGGCTGAAGGTCTACCCCATTGACCATGGGCCCTGGGGGGAGGACGAG
GAGTGGACAGACAAGGCCCGGCGGGTCATCATGGAGCGTATCGGCCTCGC
CACTGCAGGGGAGCCCTACCACGACATCCGCTTCAACCTGATGGCAGTGG
TGCCCGACCGCAGGATCAAGTATGAGGCCAGGCTGCATGTGCTGAAGGTG
AACCGTCAGACAGTACTAGAGGCTCTGCAGCAGCTGATAAGAGTAACACA
GCCAGAGCTGATTCAGACCCACAAGTCTCAAGAGTCACAGCTGCCTGAGG
AGTCCAAGTCAGCCAGCAACAAGTCCCCGCTGGTGCTGGAAGCAAACAGG
GCCCCTGCAGCCTCTGAGGGCAACCACACAGATGGTGCAGAGGAGGCGGC
TGGTTCATGCGCACAAGCCCCATCCCACAGCCCTCCCAACAAACCCAAGC
TAGTGGTGAAGCCTCCAGGCAGCAGCCTCAATGGGGTTCACCCCAACCCC
ACTCCCATTGTCCAGCGGCTGCCGGCCTTTCTAGACAATCACAATTATGC
CAAGTCCCCCATGCAGGAGGAAGAAGACCTGGCGGCAGGTGTGGGCCGCA
GCCGAGTTCCAGTCCGCCCACCCCAGCAGTACTCAGATGATGAGGATGAC
TATGAGGATGACGAGGAGGATGACGTGCAGAACACCAACTCTGCCCTTAG
GTATAAGGGGAAGGGAACAGGGAAGCCAGGGGCATTGAGCGGTTCTGCTG
ATGGGCAACTGTCAGTGCTGCAGCCCAACACCATCAACGTCTTGGCTGAG
AAGCTCAAAGAGTCCCAGAAGGACCTCTCAATTCCTCTGTCCATCAAGAC
TAGCAGCGGGGCTGGGAGTCCGGCTGTGGCAGTGCCCACACACTCGCAGC
CCTCACCCACCCCCAGCAATGAGAGTACAGACACGGCCTCTGAGATCGGC
AGTGCTTTCAACTCGCCACTGCGCTCGCCTATCCGCTCAGCCAACCCGAC
GCGGCCCTCCAGCCCTGTCACCTCCCACATCTCCAAGGTGCTTTTTGGAG
AGGATGACAGCCTGCTGCGTGTTGACTGCATACGCTACAACCGTGCTGTC
CGTGATCTGGGTCCTGTCATCAGCACAGGCCTGCTGCACCTGGCTGAGGA
TGGGGTGCTGAGTCCCCTGGCGCTGACAGAGGGTGGGAAGGGTTCCTCGC
CCTCCATCAGACCAATCCAAGGCAGCCAGGGGTCCAGCAGCCCAGTGGAG
AAGGAGGTCGTGGAAGCCACGGACAGCAGAGAGAAGACGGGGATGGTGAG
GCCTGGCGAGCCCTTGAGTGGGGAGAAATACTCACCCAAGGAGCTGCTGG
CACTGCTGAAGTGTGTGGAGGCTGAGATTGCAAACTATGAGGCGTGCCTC
AAGGAGGAGGTAGAGAAGAGGAAGAAGTTCAAGATTGATGACCAGAGAAG
GACCCACAACTACGATGAGTTCATCTGCACCTTTATCTCCATGCTGGCTC
AGGAAGGCATGCTGGCCAACCTAGTGGAGCAGAACATCTCCGTGCGGCGG
CGCCAAGGGGTCAGCATCGGCCGGCTCCACAAGCAGCGGAAGCCTGACCG
GCGGAAACGCTCTCGCCCCTACAAGGCCAAGCGCCAGTGA

The amino acid sequence of one embodiment of human wild-type BAP1 is referred to herein as SEQ ID No. 3, as follows:

[SEQ ID No. 3]
MNKGWLELESDPGLFTLLVEDFGVKGVQVEEIYDLQSKCQGPVYGFIFLF
KWIEERRSRRKVSTLVDDTSVIDDDIVNNMFFAHQLIPNSCATHALLSVL
LNCSSVDLGPTLSRMKDFTKGFSPESKGYAIGNAPELAKAHNSHARPEPR
HLPEKQNGLSAVRTMEAFHFVSYVPITGRLFELDGLKVYPIDHGPWGEDE
EWTDKARRVIMERIGLATAGEPYHDIRFNLMAVVPDRRIKYEARLHVLKV
NRQTVLEALQQLIRVTQPELIQTHKSQESQLPEESKSASNKSPLVLEANR
APAASEGNHTDGAEEAAGSCAQAPSHSPPNKPKLVVKPPGSSLNGVHPNP
TPIVQRLPAFLDNHNYAKSPMQEEEDLAAGVGRSRVPVRPPQQYSDDEDD
YEDDEEDDVQNTNSALRYKGKGTGKPGALSGSADGQLSVLQPNTINVLAE
KLKESQKDLSIPLSIKTSSGAGSPAVAVPTHSQPSPTPSNESTDTASEIG
SAFNSPLRSPIRSANPTRPSSPVTSHISKVLFGEDDSLLRVDCIRYNRAV
RDLGPVISTGLLHLAEDGVLSPLALTEGGKGSSPSIRPIQGSQGSSSPVE
KEVVEATDSREKTGMVRPGEPLSGEKYSPKELLALLKCVEAEIANYEACL
KEEVEKRKKFKIDDQRRTHNYDEFICTFISMLAQEGMLANLVEQNISVRR
RQGVSIGRLHKQRKPDRRKRSRPYKAKRQ

Tumours arise due to mutations in proto-oncogenes or tumour suppressor genes. A gain-of-function mutation in a proto-oncogene converts it into an oncogene, which causes tumourigenesis. Such gain-of-function mutations are usually missense mutations in the DNA bases, which confer a change in the amino acid sequence, or a gain of copy number of the gene. A loss-of-function mutation in a tumour suppressor gene will also lead to tumourigenesis. A loss-of-function mutation in a tumour suppressor however is not restricted to the change of a few amino acids. Any change in an amino acid which impairs the function of the protein, results in a loss-of-function mutation. Hence, unlike oncogenes, it is not always possible to identify a specific mutation which results in the loss of function of a tumour suppressor gene. Loss-of-function truncating mutations of tumour suppressors are therefore typically identified throughout the coding exons of a gene.

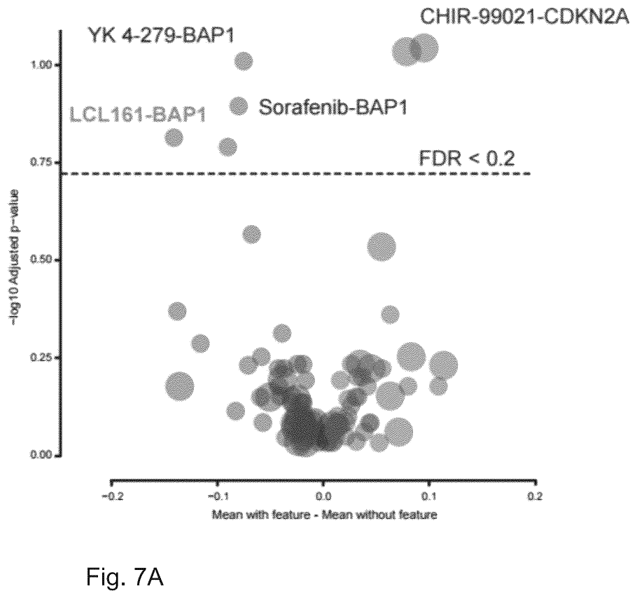

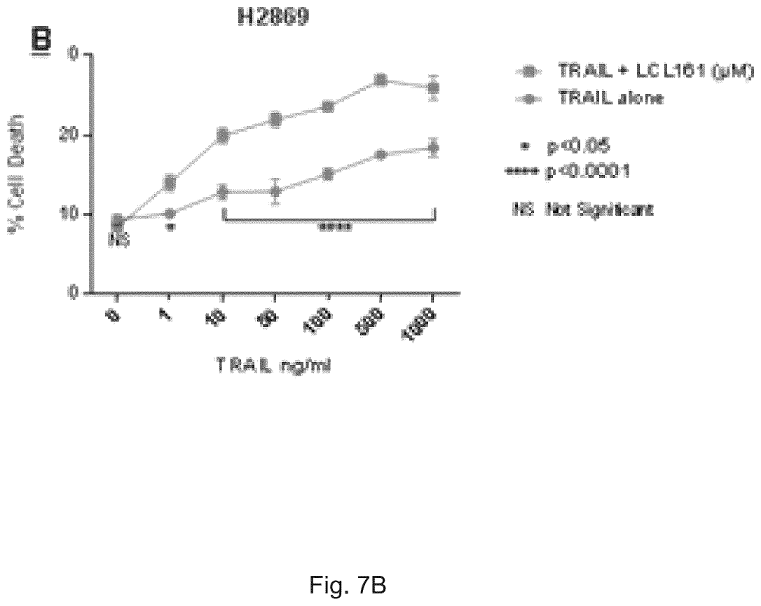

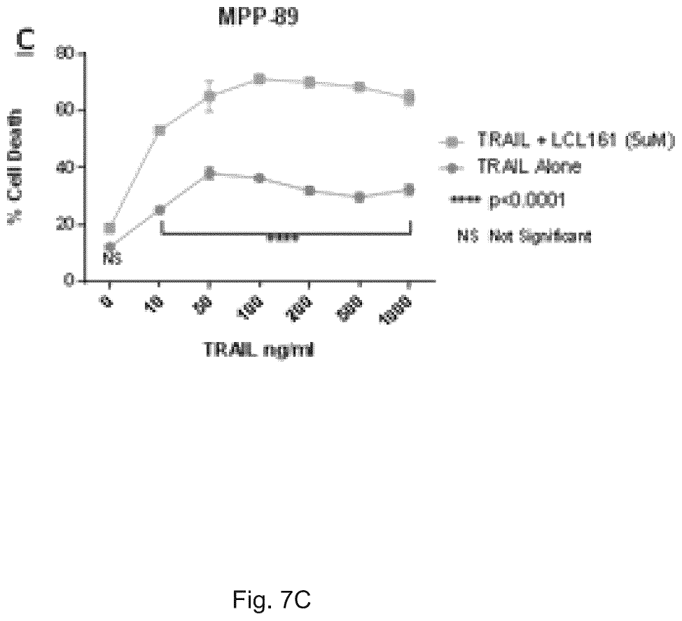

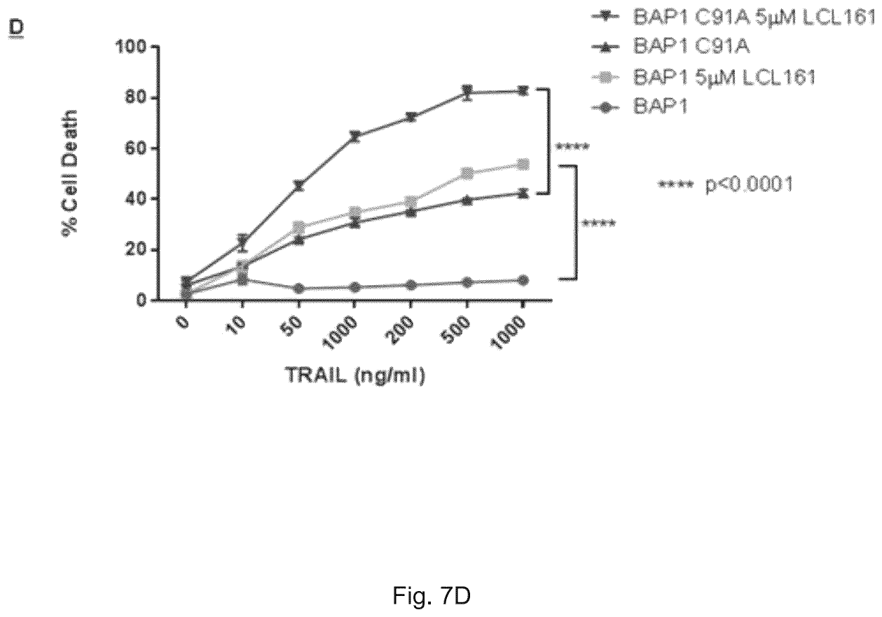

BAP1 is a tumour suppressor gene. According to the invention, a mutant BAP1 gene is one that comprises a mutation. The mutant BAP1 gene is a gene that encodes a non-functional or enzymatically inactive BAP1 protein, or a BAP1 protein that exhibits reduced binding to an ASXL protein compared to the level of binding in a reference cell, which is resistant to DRL-induced cell death. Thus, a mutant BAP1 protein is one that is non-functional or enzymatically inactive or incapable of binding to an ASXL protein or exhibits reduced binding to an ASXL protein compared to the level of binding in a reference cell, which is resistant to DRL-induced cell death. As described in the Examples (see FIG. 7d), BAP1 has surprisingly been shown to form a complex with ASXL1, ASXL2 or ASXL3. These proteins will be collectively referred to herein as an ASXL protein. BAP1-ASXL complexes have been shown to deubiquitinate Histone 2A, and other substrates. Inhibiting the formation of this complex renders BAP1 non-functional or enzymatically inactive.

The mutant BAP1 protein may be a full-length protein with mutations at specific loci. The mutant BAP1 protein may be a partial or complete deletion or mutation of the wild-type BAP1 protein. Partial deletion or mutation may occur in the nuclear localisation sequence (NLS), the active site of wild-type BAP1, the binding site of ASXL, or at any place in the gene that would result in the loss of function of BAP1. Mutation may include one or more point mutations. Point mutations may be a substitution, an insertion, a deletion or a frameshift mutation.

A reduced level of expression of a wild-type BAP1 gene compared to the level of expression in a reference cell may result in a lower wild-type BAP1 protein concentration compared to the protein concentration in the same reference cell. The reduced level of expression of the wild-type BAP1 gene may be at least a 10%, 15%, 25%, 35%, 45%, 50%, 55%, 60%, 65%, 70%, 75%, 80%, 85%, 90%, 95% or a 100% reduction compared to the reference cell. Similarly, the lower concentration of wild-type BAP1 protein may be lower by at least 10%, 15%, 25%, 35%, 45%, 50%, 55%, 60%, 65%, 70%, 75%, 80%, 85%, 90%, 95% or 100% compared to the reference cell. The skilled person would know how to detect the extent of the reduction in BAP1 gene expression or lower BAP1 protein expression.

Lower expression of wild-type BAP1 protein may be caused by epigenetic silencing, methylation or low levels of BAP1 gene expression. The term “non-functional BAP1 protein” can refer to but is not limited to a BAP1 protein that does not exhibit deubiquitinase (enzyme) activity. The skilled person would appreciate that standard assays for measuring deubiquitinase activity include, but are not limited to, fluorescence assays using a fluorogenic substrate, such as ubiquitin-amidomethylcoumarin; and HPLC assays using ubiquitin ethyl ester or ubiquitin fusion peptides as model substrates to monitor deubiquitinating activity.

In one embodiment, the amino acid sequence of a mutant BAP1 gene (p.R60Q) is referred to herein as SEQ ID No. 4, as follows:

| [SEQ ID No. 4] | |
|---|---|
| 1 | mnkgwleles dpglftllve dfgvkgvqve eiydlqskcq gpvygfiflf kwieerrsrq |
| 61 | kvstlvddts vidddivnnm ffahqlipns cathallsvl lncssvdlgp tlsrmkdftk |
| 121 | gfspeskgya ignapelaka hnsharpepr hlpekqngls avrtmeafhf vsyvpitgrl |
| 181 | feldglkvyp idhgpwgede ewtdkarrvi meriglatag epyhdirfnl mavvpdrrik |
| 241 | yearlhvlkv nrqtvlealq qlirvtqpel iqthksqesq lpeesksasn ksplvleanr |
| 301 | apaasegnht dgaeeaagsc aqapshsppn kpklvvkppg sslngvhpnp tpivqrlpaf |
| 361 | ldnhnyaksp mqeeedlaag vgrsrvpvrp pqqysddedd yeddeeddvq ntnsalrykg |
| 421 | kgtgkpgals gsadgqlsvl qpntinvlae klkesqkdls iplsiktssg agspavavpt |
| 481 | hsqpsptpsn estdtaseig safnsplrsp irsanptrps spvtshiskv lfgeddsllr |
| 541 | vdcirynrav rdlgpvistg llhlaedgvl splalteggk gsspsirpiq gsqgssspve |
| 601 | kevveatdsr ektgmvrpge plsgekyspk ellallkcve aeianyeacl keevekrkkf |
| 661 | kiddqrrthn ydefictfis mlaqegmlan lveqnisvrr rqgvsigrlh kqrkpdrrkr |
| 721 | srpykakrq |

Therefore, the amino acid sequence of the mutant BAP1 protein may be encoded by SEQ ID NO. 4 or a fragment or variant thereof.

In one embodiment, the nucleotide sequence of a mutant BAP1 gene is referred to herein as SEQ ID No. 5, as follows:

[SEQ ID No. 5]
ATGAATAAGGGCTGGCTGGAGCTGGAGAGCGACCCAGGCCTCTTCACCCT
GCTCGTGGAAGATTTCGGTGTCAAGGGGGTGCAAGTGGAGGAGATCTACG
ACCTTCAGAGCAAATGTCAGGGCCCTGTATATGGATTTATCTTCCTGTTC
AAATGGATCGAAGAGCGCCGGTCCCGGCAAAAGGTCTCTACCTTGGTGGA
TGATACGTCCGTGATTGATGATGATATTGTGAATAACATGTTCTTTGCCC
ACCAGCTGATACCCAACTCTTGTGCAACTCATGCCTTGCTGAGCGTGCTC
CTGAACTGCAGCAGCGTGGACCTGGGACCCACCCTGAGTCGCATGAAGGA
CTTCACCAAGGGTTTCAGCCCTGAGAGCAAAGGATATGCGATTGGCAATG
CCCCGGAGTTGGCCAAGGCCCATAATAGCCATGCCAGGCCCGAGCCACGC
CACCTCCCTGAGAAGCAGAATGGCCTTAGTGCAGTGCGGACCATGGAGGC
GTTCCACTTTGTCAGCTATGTGCCTATCACAGGCCGGCTCTTTGAGCTGG
ATGGGCTGAAGGTCTACCCCATTGACCATGGGCCCTGGGGGGAGGACGAG
GAGTGGACAGACAAGGCCCGGCGGGTCATCATGGAGCGTATCGGCCTCGC
CACTGCAGGGGAGCCCTACCACGACATCCGCTTCAACCTGATGGCAGTGG
TGCCCGACCGCAGGATCAAGTATGAGGCCAGGCTGCATGTGCTGAAGGTG
AACCGTCAGACAGTACTAGAGGCTCTGCAGCAGCTGATAAGAGTAACACA
GCCAGAGCTGATTCAGACCCACAAGTCTCAAGAGTCACAGCTGCCTGAGG
AGTCCAAGTCAGCCAGCAACAAGTCCCCGCTGGTGCTGGAAGCAAACAGG
GCCCCTGCAGCCTCTGAGGGCAACCACACAGATGGTGCAGAGGAGGCGGC
TGGTTCATGCGCACAAGCCCCATCCCACAGCCCTCCCAACAAACCCAAGC
TAGTGGTGAAGCCTCCAGGCAGCAGCCTCAATGGGGTTCACCCCAACCCC
ACTCCCATTGTCCAGCGGCTGCCGGCCTTTCTAGACAATCACAATTATGC
CAAGTCCCCCATGCAGGAGGAAGAAGACCTGGCGGCAGGTGTGGGCCGCA
GCCGAGTTCCAGTCCGCCCACCCCAGCAGTACTCAGATGATGAGGATGAC
TATGAGGATGACGAGGAGGATGACGTGCAGAACACCAACTCTGCCCTTAG
GTATAAGGGGAAGGGAACAGGGAAGCCAGGGGCATTGAGCGGTTCTGCTG
ATGGGCAACTGTCAGTGCTGCAGCCCAACACCATCAACGTCTTGGCTGAG
AAGCTCAAAGAGTCCCAGAAGGACCTCTCAATTCCTCTGTCCATCAAGAC
TAGCAGCGGGGCTGGGAGTCCGGCTGTGGCAGTGCCCACACACTCGCAGC
CCTCACCCACCCCCAGCAATGAGAGTACAGACACGGCCTCTGAGATCGGC
AGTGCTTTCAACTCGCCACTGCGCTCGCCTATCCGCTCAGCCAACCCGAC
GCGGCCCTCCAGCCCTGTCACCTCCCACATCTCCAAGGTGCTTTTTGGAG
AGGATGACAGCCTGCTGCGTGTTGACTGCATACGCTACAACCGTGCTGTC
CGTGATCTGGGTCCTGTCATCAGCACAGGCCTGCTGCACCTGGCTGAGGA
TGGGGTGCTGAGTCCCCTGGCGCTGACAGAGGGTGGGAAGGGTTCCTCGC
CCTCCATCAGACCAATCCAAGGCAGCCAGGGGTCCAGCAGCCCAGTGGAG
AAGGAGGTCGTGGAAGCCACGGACAGCAGAGAGAAGACGGGGATGGTGAG
GCCTGGCGAGCCCTTGAGTGGGGAGAAATACTCACCCAAGGAGCTGCTGG
CACTGCTGAAGTGTGTGGAGGCTGAGATTGCAAACTATGAGGCGTGCCTC
AAGGAGGAGGTAGAGAAGAGGAAGAAGTTCAAGATTGATGACCAGAGAAG
GACCCACAACTACGATGAGTTCATCTGCACCTTTATCTCCATGCTGGCTC
AGGAAGGCATGCTGGCCAACCTAGTGGAGCAGAACATCTCCGTGCGGCGG
CGCCAAGGGGTCAGCATCGGCCGGCTCCACAAGCAGCGGAAGCCTGACCG
GCGGAAACGCTCTCGCCCCTACAAGGCCAAGCGCCAGTGA

Therefore, the nucleotide sequence of the mutant BAP1 gene may be encoded by SEQ ID NO. 5 or a fragment or variant thereof.

Reduced or non-binding between an ASXL protein (ASXL1, ASXL2 or ASXL3) and a wild-type BAP1 protein may be at least a 10%, 15%, 25%, 35%, 45%, 50%, 55%, 60%, 65%, 70%, 75%, 80%, 85%, 90%, 95% or a 100% reduction compared to a reference cell in which binding between a wild-type BAP1 protein and an ASXL protein occurs.

The skilled person would know how to detect the extent of the reduction in wild-type BAP1 protein binding to an ASXL protein. Standard assays for measuring binding between a wild-type BAP1 protein and an ASXL protein include but are not limited to protein complex immunoprecipitation or fluorescence resonance energy transfer (FRET).

The term “DRL-induced cell death” can refer to, but is not limited to, apoptosis and other types of cell death caused by a death receptor ligand (DRL), such as necroptosis and necrosis. Thus, the term “death receptor ligand” refers to any agent that binds to a cellular receptor and induces death of the cell on which the receptor is located. The term “cell death” can refer to cellular apoptosis, necrosis and necroptosis. Preferably, it refers to cellular apoptosis. Apoptosis refers to programmed cell death caused by activation of an apoptotic signal transduction pathway. This may be achieved through the binding of a DRL to a death receptor. Death receptor ligands may be TRAIL, TNFalpha, FAS ligand (FASL), recombinant TRAIL (dulanermin), antibodies to death receptors, especially antibodies to death receptors of the ligand TRAIL (such as mapatumuab, drozitumumab, conatumumab, lexatumumab, tigatuzumab, LBY-135), or a combination thereof.

The DRL may be an agonist molecule such as Medi-3039 or any agent that activates an apoptotic signal transduction pathway. An extrinsic apoptotic signal transduction pathway may be the FAS ligand pathway, the TNFalpha pathway or the TRAIL pathway.

The term “express(ed) or expression” can refer to a transcribed gene (i.e. DNA), or corresponding RNA that has been translated into a polypeptide or protein. Expression of a BAP1 mutant gene or BAP1 polypeptide may be detected in any compartment of the cell (e.g. in the nucleus, cytosol, the Endoplasmic Reticulum, the Golgi apparatus or the intracellular surface of the plasma membrane).

Detecting according to (i), (ii) or (iii) of the first aspect may comprise the use of any one of the following assays for detecting the presence of a gene or its corresponding protein in a sample: polymerase chain reaction (PCR); northern blotting; hybridisation-based detection techniques; flow cytometry; immunoassays, such as enzyme-linked immunosorbent assays (ELISAs), an enzyme immunoassay (EIAs), radioimmunoassay (RIAs), Western Blots, immuno-precipitation or immunohistochemistry; immunofluorescence; chromogenic (enzyme activity) assays; fluorometric imaging plate reader (FLIPR) assay; high performance liquid chromatography (HPLC) tandem mass spectrometry (MS/MS).

The biological sample is preferably a cancerous bodily sample taken from a test individual. Detection for the presence of a BAP1 mutant gene or mutant BAP1 protein in the sample is therefore preferably carried out in vitro. The sample may comprise tissue, blood, plasma, serum, spinal fluid, urine, sweat, saliva, sputum, tears, breast aspirate, prostate fluid, seminal fluid, vaginal fluid, stool, cervical scraping, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The sample may be a biopsy.

In another embodiment, the sample may be contained within the test subject, which may be an experimental animal (e.g. a mouse or rat) or a human, wherein the method is an in vivo based test. Alternatively, the sample may be an ex vivo sample or an in vitro sample. Therefore, the cells being tested may be in a tissue sample (for ex vivo based tests) or the cells may be grown in culture (an in vitro sample). Preferably, the biological sample is an ex vivo sample.

The inventors have developed a prognostic kit for determining a subject's sensitivity or otherwise to DRL-induced cell death.

According to a second aspect, there is provided a kit for determining if an individual's cancer cell is sensitive to DRL-induced cell death, the kit comprising detection means for detecting the expression of a mutant BAP1 gene or mutant BAP1 protein, or for detecting a reduced level of expression of a wild-type BAP1 gene or a lower wild-type BAP1 protein concentration compared to the level of expression or protein concentration in a reference cell that is resistant to DRL-induced cell death, or for detecting non-binding or reduced binding of an ASXL protein to a BAP1 protein compared to the level of binding in a reference cell that is a BAP1 wild-type cell that is resistant to DRL-induced cell death, wherein the presence, in the sample, of the mutant BAP1 gene or the BAP1 protein, or of a reduced level expression of the wild-type BAP1 gene or a lower wild-type BAP1 protein concentration, or reduced or non-binding of an ASXL protein to a wild-type BAP1 protein, is indicative of the individual's cancer cell being sensitive to DRL-induced cell death.

Preferably, the kit is used to provide a prognosis for an individual being treated with a death receptor ligand (DRL) or any agents activating the apoptotic pathways. A death receptor ligand may be TRAIL, TNFalpha, FAS ligand (FASL), recombinant TRAIL (dulanermin), death receptor antibodies (such as mapatumuab, drozitumumab, conatumumab, lexatumumab, tigatuzumab), death receptor agonists, such as Medi-3038 or Medi-3039, or a combination thereof.

Preferably, the kit comprises at least one control or reference sample. The kit may comprise a negative control and/or a positive control. A negative control may comprise a wild-type BAP1 protein that is resistant to DRL-induced cell death. A positive control may comprise a mutant BAP1 mRNA, or mutant BAP1 protein, or a blank sample. The skilled person will appreciate that the level of mRNA in a sample is indicative of the level of gene expression in a cell.

The detection means is preferably configured to detect the expression or the concentration of a mutant BAP1 protein or mRNA, or wild-type BAP1 mRNA or BAP1 protein in the biological sample taken from the test individual. The presence of the mutant BAP1 mRNA or mutant BAP1 protein, or reduced level of expression of wild-type BAP1 mRNA or lower protein expression to the control, in the sample, is indicative that the test sample is sensitive to DRL-induced cell death. The level of expression of the wild-type BAP1 mRNA or concentration of BAP1 protein in the biological sample may be reduced or lower compared to the concentration or level of expression of the wild-type BAP1 mRNA or protein in a negative control. The reduction in expression may be at least a 10%, 15%, 25%, 35%, 45%, 50%, 55%, 60%, 65%, 70%, 75%, 80%, 85%, 90%, 95% or 100% reduction compared to the negative control. Similarly, the concentration of the wild-type BAP1 protein in the biological sample may be lower by at least a 10%, 15%, 25%, 35%, 45%, 50%, 55%, 60%, 65%, 70%, 75%, 80%, 85%, 90%, 95% or 100%. Conversely, the absence of a mutant BAP1 gene or protein, or normal to high expression of the wild-type BAP1 gene or protein, in the sample, is indicative that the sample is insensitive to DRL-induced cell death.

Detection of a mutation in a BAP1 gene or protein can be achieved using a number of sequencing approaches. In one approach, whole exome or targeted gene sequencing is undertaken with massively parallel sequencing of tumour DNA with target genes enriched using commercially available RNA baits.

In another embodiment, a capillary sequencing approach is utilised with PCR primers designed to each of the exons in the BAP1 gene footprint. Thus, the detection means may be a primer.

In one embodiment, the nucleic acid sequence of a forward primer used to detect exon 12 (thr3:52438255-52438817F) of wild-type BAP1 mRNA is referred to herein as SEQ ID No. 6 as follows:

[SEQ ID No. 6]

acctagaacctggtagccttag

In one embodiment, the nucleic acid sequence of a forward primer used to detect exon 8 (chr3:52440631-52441138F) of wild-type BAP1 mRNA is referred to herein as SEQ ID No. 7 as follows:

[SEQ ID No. 7]

gtacagctccagagagtagaac

In one embodiment, the nucleic acid sequence of a forward primer used to detect exon 1 (chr3:52443644-52444094F) of wild-type BAP1 mRNA is referred to herein as SEQ ID No. 8 as follows:

[SEQ ID No. 8]

tcttaccgaaatcttccacgag

In one embodiment, the nucleic acid sequence of a forward primer used to detect exon 3 (chr3:52443356-52443839F) of wild-type BAP1 mRNA is referred to herein as SEQ ID No. 9 as follows:

[SEQ ID No. 9]

ctgctgctttctgtgagatttt

In one embodiment, the nucleic acid sequence of a forward primer used to detect exon 6 (chr3:52436404-52436905F) of wild-type BAP1 mRNA is referred to herein as SEQ ID No. 10 as follows:

[SEQ ID No. 10]

agggcattccagttaagacag

In one embodiment, the nucleic acid sequence of a forward primer used to detect exon 17 (chr3:52436105-52436652F) of wild-type BAP1 mRNA is referred to herein as SEQ ID No. 11 as follows:

[SEQ ID No. 11]

caagagtgggctgcagag

In one embodiment, the nucleic acid sequence of a forward primer used to detect exon 6 (chr3:52441201-52441691F) of wild-type BAP1 mRNA is referred to herein as SEQ ID No. 12 as follows:

[SEQ ID No. 12]

actaaggccattctgcttctc

In one embodiment, the nucleic acid sequence of a forward primer used to detect exon 4 (chr3:52442276-52442837F) of wild-type BAP1 mRNA is referred to herein as SEQ ID No. 13 as follows:

[SEQ ID No. 13]

atcccaccctccaaacaaag

In one embodiment, the nucleic acid sequence of a forward primer used to detect exon 13a (chr3:52437218-52437786F) of wild-type BAP1 mRNA is referred to herein as SEQ ID No. 14 as follows:

[SEQ ID No. 14]

caccaagtggccagtgag

In one embodiment, the nucleic acid sequence of a forward primer used to detect exon 13b (chr3:52437388-52437956F) of wild-type BAP1 mRNA is referred to herein as SEQ ID No. 15 as follows:

[SEQ ID No. 15]

ggctgtcatcctctccaaaa

In one embodiment, the nucleic acid sequence of a forward primer used to detect exon 13c (chr3:52437558-52438125F) of wild-type BAP1 mRNA is referred to herein as SEQ ID No. 16 as follows:

[SEQ ID No. 16]

gagggctgcgagtgtgtg

In one embodiment, the nucleic acid sequence of a forward primer used to detect exon 14 (chr3:52436940-52437529F) of wild-type BAP1 mRNA is referred to herein as SEQ ID No. 17 as follows:

[SEQ ID No. 17]

ctctgccaggattaaaggagaa

In one embodiment, the nucleic acid sequence of a forward primer used to detect exon 9 (chr3:52440055-52440607F) of wild-type BAP1 mRNA is referred to herein as SEQ ID No. 18 as follows:

[SEQ ID No. 18]

gaatgcagggagggttgg

In one embodiment, the nucleic acid sequence of a forward primer used to detect exon 5 (chr3:52441760-52442308F) of wild-type BAP1 mRNA is referred to herein as SEQ ID No. 19 as follows:

[SEQ ID No. 19]

acccaatatcatgtggtagcat

In one embodiment, the nucleic acid sequence of a forward primer used to detect exon 2 (Chr3:52443516-52443974F) of wild-type BAP1 mRNA is referred to herein as SEQ ID No. 20 as follows:

[SEQ ID No. 20]

aaggacagcccctgatga

In one embodiment, the nucleic acid sequence of a forward primer used to detect exon 7 (chr3:52440976-52441547F) of wild-type BAP1 mRNA is referred to herein as SEQ ID No. 21 as follows:

[SEQ ID No. 21]

gtaggcagagacacccaac

In one embodiment, the nucleic acid sequence of a forward primer used to detect exon 15 (chr3:52436581-52437102F) of wild-type BAP1 mRNA is referred to herein as SEQ ID No. 22 as follows:

[SEQ ID No. 22]

ccttctctggtcatcaatctgt

In one embodiment, the nucleic acid sequence of a forward primer used to detect exon 10 (chr3:52439567-52440143F) of wild-type BAP1 mRNA is referred to herein as SEQ ID No. 23 as follows:

[SEQ ID No. 23]

ctctgaggtccacaagaggt

In one embodiment, the nucleic acid sequence of a forward primer used to detect exon 11 (chr3:52438912-52439525F) of wild-type BAP1 mRNA is referred to herein as SEQ ID No. 24 as follows:

[SEQ ID No. 24]

tcaagtagagaatcctgcaagg

In one embodiment, the nucleic acid sequence of a reverse primer used to detect exon 12 (Chr3:52438255-52438817R) of wild-type BAP1 mRNA is referred to herein as SEQ ID No. 25 as follows:

[SEQ ID No. 25]

gagcagcacttgtttgtaactg

In one embodiment, the nucleic acid sequence of a reverse primer used to detect exon 8 (chr3:52440631-52441138R) of wild-type BAP1 mRNA is referred to herein as SEQ ID No. 26 as follows:

[SEQ ID No. 26]

ctcaactgctcttctagtctt

In one embodiment, the nucleic acid sequence of a reverse primer used to detect exon 1 (chr3:52443644-52444094R) of wild-type BAP1 mRNA is referred to herein as SEQ ID No. 27 as follows:

[SEQ ID No. 27]

gagggagggcctggacat

In one embodiment, the nucleic acid sequence of a reverse primer used to detect exon 3 (chr3:52443356-52443839R) of wild-type BAP1 mRNA is referred to herein as SEQ ID No. 28 as follows:

[SEQ ID No. 28]

ctgtccttccctactgctttc

In one embodiment, the nucleic acid sequence of a reverse primer used to detect exon 16 (chr3:52436404-52436905R) of wild-type BAP1 mRNA is referred to herein as SEQ ID No. 29 as follows:

[SEQ ID No. 29]

gaagttcaaggtgggtgatttc

In one embodiment, the nucleic acid sequence of a reverse primer used to detect exon 17 (chr3:52436105-52436652R) of wild-type BAP1 mRNA is referred to herein as SEQ ID No. 30 as follows:

[SEQ ID No. 30]

ctcagctcctggcctgag

In one embodiment, the nucleic acid sequence of a reverse primer used to detect exon 6 (chr3:52441201-52441691R) of wild-type BAP1 mRNA is referred to herein as SEQ ID No. 31 as follows:

[SEQ ID No. 31]

ggagaaattattctgatacggcc

In one embodiment, the nucleic acid sequence of a reverse primer used to detect exon 4 (chr3:52442276-52442837R) of wild-type BAP1 mRNA is referred to herein as SEQ ID No. 32 as follows:

[SEQ ID No. 32]

gaagggaatgctgattgtcttc

In one embodiment, the nucleic acid sequence of a reverse primer used to detect exon 13a (chr3:52437218-52437786R) of wild-type BAP1 mRNA is referred to herein as SEQ ID No. 33 as follows:

[SEQ ID No. 33]

ctatccgctcagccaacc

In one embodiment, the nucleic acid sequence of a reverse primer used to detect exon 13b (chr3:52437388-52437956R) of wild-type BAP1 mRNA is referred to herein as SEQ ID No. 34 as follows:

[SEQ ID No. 34]

ctctcaattcctctgtccatca

In one embodiment, the nucleic acid sequence of a reverse primer used to detect exon 13c (chr3:52437558-52438125R) of wild-type BAP1 mRNA is referred to herein as SEQ ID No. 35 as follows:

[SEQ ID No. 35]

cgttcccttgcttcacatct

In one embodiment, the nucleic acid sequence of a reverse primer used to detect exon 14 (chr3:52436940-52437529R) of wild-type BAP1 mRNA is referred to herein as SEQ ID No. 36 as follows:

[SEQ ID No. 36]

tcctgcactctgatgattttct

In one embodiment, the nucleic acid sequence of a reverse primer used to detect exon 9 (chr3:52440055-52440607R) of wild-type BAP1 mRNA is referred to herein as SEQ ID No. 37 as follows:

[SEQ ID No. 37]

gatatctgcctcaacctgatgg

In one embodiment, the nucleic acid sequence of a reverse primer used to detect exon 5 (chr3:52441760-52442308R) of wild-type BAP1 mRNA is referred to herein as SEQ ID No. 38 as follows:

[SEQ ID No. 38]

gtgctgtgtatgggtgacta

In one embodiment, the nucleic acid sequence of a reverse primer used to detect exon 2 (chr3:52443516-52443974R) of wild-type BAP1 mRNA is referred to herein as SEQ ID No. 39 as follows:

[SEQ ID No. 39]

gaagatgaataagggctggct

In one embodiment, the nucleic acid sequence of a reverse primer used to detect exon 7 (chr3:52440976-52441547R) of wild-type BAP1 mRNA is referred to herein as SEQ ID No. 40 as follows:

[SEQ ID No. 40]

tgatgtggggtgggagtag

In one embodiment, the nucleic acid sequence of a reverse primer used to detect exon (chr3:52436581-52437102R) of wild-type BAP1 mRNA is referred to herein as SEQ ID No. 41 as follows:

[SEQ ID No. 41]

cccgatcagaggtgcaat

In one embodiment, the nucleic acid sequence of a reverse primer used to detect exon 10 (chr3:52439567-52440143R) of wild-type BAP1 mRNA is referred to herein as SEQ ID No. 42 as follows:

[SEQ ID No. 42]

agctatttaaggtagaagcccg

In one embodiment, the nucleic acid sequence of a reverse primer used to detect exon 11 (chr3:52438912-52439525R) of wild-type BAP1 mRNA is referred to herein as SEQ ID No. 43 as follows:

[SEQ ID No. 43]

actgtgagcttttcttggagat

The skilled person would appreciate that the binding of an ASXL protein to a wild-type BAP1 protein may be achieved using a variety of techniques known in the art, which include protein complex immunoprecipitation, Bimolecular Fluorescence complementation, Affinity electrophoresis, Immunoelectrophoresis, chemical cross linking, Proximity ligation assay and FRET.

The inventors believe that their findings (i.e. that cells containing a mutant BAP1 gene or a mutant BAP1 protein, or cells expressing reduced levels of a wild-type BAP1 gene or with a lower concentration of BAP1 protein, are sensitive to DRL-induced cell death) means that they have identified a robust biomarker for sensitivity to DRL-induced cell death.

Therefore, in a third aspect of the invention, there is provided a use of (i) a mutant BAP1 gene or a mutant BAP1 protein, or (ii) a cancer cell with a reduced level of expression of a wild-type BAP1 gene or a lower wild-type BAP1 protein concentration compared to the level of expression or protein concentration in a reference cell that is a BAP1 wild-type cell, as a biomarker of sensitivity to DRL-induced cell death.

The inventors have developed a method of treating subjects suffering from cancer, and especially those suffering from a cancer that is completely or partially insensitive to death receptor ligand-induced cell death.

In a fourth aspect, therefore, there is provided a method of treating an individual suffering from cancer, the method comprising:
(i) detecting for the presence of a mutant BAP1 gene or mutant BAP1 protein, or for a reduced level of expression of a wild-type BAP1 gene or a lower wild-type BAP1 protein concentration compared to the level of expression or protein concentration in a reference cell that is a BAP1 wild-type cell that is resistant to DRL-induced cell death, or for reduced or non-binding of an ASXL protein to a wild-type BAP1 protein compared to the level of binding in a reference cell that is a BAP1 wild-type cell, which is resistant to DRL-induced cell death; and
(ii) administering a therapeutically effective amount of a death receptor ligand to the individual.

The death receptor ligand may be administered as a monotherapy or in combination with other agents that are capable of killing cancer cells.

The “subject” or “individual” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

As mentioned above, mesothelioma cells are resistant to the induction of cell death. This disruption of core-cell death machinery has been implicated in the resistance to conventional cytotoxic agents generally observed clinically. It is believed that disruption of cell death machinery is mediated downstream through elevated expression of anti-cell death proteins such as members of the IAP (inhibitors of apoptosis) family or the BCL-2 family. The IAP family consist of 8 members (BIRC2, BIRC3, BIRC5, BIRC6, BIRC7, BIRC8, NAIP, XIAP). The defining feature of an IAP protein is the presence of a ˜70-amino acid baculovirus IAP repeat (BIR) domain that mediates protein-protein interactions (12). Through these domains, IAP members act as endogenous inhibitors of caspases, the main executioners of cell death, and act through either direct caspase inhibition or ubiquitin-mediated regulation of caspase degradation (13).

The inventors have demonstrated that BAP1 gene expression can be reduced in mesothelioma cell lines using a BAP1 shRNA-expressing lentivirus and that this reduction of BAP1 gene expression results in increased sensitivity to DLR-induced cell death in these cell lines. Accordingly, the inventors have determined that it is possible to sensitise to DRL-induced cell death, an individual suffering from a cancer that is normally insensitive to DRL-induced cell death, by administering a BAP1 inhibitor.

Thus, in a fifth aspect of the invention, there is provided a method of:
sensitising, to DRL-induced cell death, an individual that is suffering from a cancer that is insensitive to DRL-induced cell death, or
enhancing the sensitivity to DRL-induced cell death in an individual that is suffering from a cancer that is sensitive to DRL-induced cell death,
the method comprising administering, to the individual, a BAP1 inhibitor or an agent that mimics the effect of BAP1 inhibition.

Thus, a BAP1 inhibitor is any agent that targets the BAP1 gene or protein directly, whereas an agent that mimics the effect of BAP1 inhibition is a molecule that targets a signalling molecule downstream of the BAP1 gene/protein signalling pathway.

A BAP1 inhibitor may be any molecule that inactivates BAP1 protein function, such as a deubiquitinase inhibitor or antagonist, or which silences or reduces BAP1 gene or protein expression or function (such as a small molecule inhibitor of BAP1), or which prevents or reduces binding of a wild-type BAP1 protein to an ASXL protein. The BAP1 inhibitor or agent may also be any molecule that mutates a wild-type BAP1 gene to create a mutant BAP1 gene. Thus, the BAP1 inhibitor or agent may be an RNAi molecule, including shRNA, siRNA, miRNA, ribozymes and antisense molecules; a TALEN (Transcriptional Activator Like-Effector Nuclease); or a CRISPR/CAS9 nuclease. The inventors have demonstrated in the Examples below that BAP1 gene expression can be reduced in mesothelioma cell lines using shRNA expressing lentivirus and that this reduction of BAP1 gene expression results in increased sensitivity to DLR-induced cell death in these cell lines.

In addition to a BAP1 inhibitor, an agent that mimics the effect of BAP1 inhibition is an agent that targets signalling or effector molecules downstream of BAP1, such agents may be used to induce DLR resistance in cancer cell. In one embodiment, the BAP1 inhibitor may be an IAP inhibitor (a SMAC mimetic). An IAP may be BIRC2, BIRC3, BIRC5, BIRC6, BIRC7, BIRC8, NAIP, XIAP or a fragment thereof. The inventors have found that BAP1 expression increases the expression of BIRC3 protein and that inhibition of BIRC3 protein with an IAP inhibitor, such as LCIA61, results in sensitization to DRL induced cell death. An IAP inhibitor is an agent that reduces expression or translation of an IAP, or renders an IAP functionally inactive. The IAP inhibitor or agent may also be any molecule that mutates an IAP gene or protein to create a mutant IAP gene or protein. Thus, the IAP1 inhibitor or agent may be an RNAi molecule, including shRNA, siRNA, miRNA, ribozymes and antisense molecules; a TALEN (Transcriptional Activator Like-Effector Nuclease); or a CRISPR/CAS9 nuclease. An agent that mimics the effect of BAP1 inhibition may be an RNA helicase inhibitor, such as, YK-4279 or a tyrosine kinase inhibitor, such as sorafenib.

The inventors believe that their surprising observation (i.e. that BAP1 plays a role in regulating DRL-induced cell death of cancer cells) can be used to develop a novel targeted approach for treatment of any cancer comprising mutants of BAP1.

Thus, in a sixth aspect of the invention, there is provided a composition comprising (i) a BAP1 inhibitor or an agent that mimics the effect of BAP1 inhibition and (ii) a death receptor ligand.

In a seventh aspect, there is provided a composition comprising (i) a BAP1 inhibitor or an agent that mimics the effect of BAP1 inhibition and (ii) a death receptor ligand, for use in therapy or as a medicament.

In an eighth aspect, there is provided a composition comprising (i) a BAP1 inhibitor or an agent that mimics the effect of BAP1 inhibition and (ii) a death receptor ligand, for use in treating, preventing or ameliorating cancer.

In a ninth aspect, there is provided a method of treating, preventing or ameliorating an individual suffering from a cancer, the method comprising administering, to the individual, a therapeutically effective amount of the composition of the sixth aspect.

The BAP1 inhibitor or agent that mimics the effect of BAP1 inhibition and the death receptor ligand may be administered simultaneously, or the BAP1 inhibitor may be administered prior to the death receptor ligand.

It will be appreciated that administration of the BAP1 inhibitor first sensitizes the subject to DRL-induced cell death. Then, administration of the death receptor ligand can be effectively used to induce cell death in cancerous cells.

Preferably, the BAP1 inhibitor is any molecule or approach which inactivates BAP1 protein function, such as a deubiquitinase inhibitor or antagonist, or which silences or reduces BAP1 gene transcription or BAP1 protein expression, or which mutates a wild-type BAP1 gene to create a BAP1 mutant gene and protein. The BAP1 inhibitor may be WP1130, Usp9x, Usp5, Usp14, Usp24, UCH37, b-AP15, CRISPR/CAS9 or a small molecule inhibitor of BAP1.

Preferred death receptor ligands may be, but are not limited to TRAIL, TNFalpha, FAS ligand (FASL), recombinant TRAIL (dulanermin), death receptor antibodies (such as mapatumuab, drozitumumab, conatumumab, lexatumumab, tigatuzumab, LBY-135) or death receptor agonists such as Medi 3038 or Medi 3039, or a combination thereof.

Mesothelioma (or Malignant Pleural Mesothelioma) is a rare form of cancer that develops from cells of the mesothelium, the protective lining that covers many of the internal organs of the body. The most common anatomical site for mesothelioma is the pleura (the outer lining of the lungs and internal chest wall), but it can also arise in the peritoneum (the lining of the abdominal cavity), the pericardium (the sac that surrounds the heart) or the tunica vaginalis (a sac that surrounds the testis). Thus, the cancer which may be treated according to any aspect of the invention may be selected from mesothelioma, Malignant Pleural Mesothelioma, uveal melanoma, melanoma, non-melanoma skin cancer, renal cancers, cholangiocarcinomas, lung cancers, cancer of the pleura, abdominal cancer, peritoneal cancer, cancer of the pericardium, head and neck cancers, brain cancers, breast cancers, liver and biliary tract cancers, gastrointestinal cancers including upper and lower tracts, urothelial cancers, prostate cancers, testicular cancer, cancer of the tunica vaginalis, ovarian cancers, cervical cancers, sarcomas, lymphomas and leukaemia.

More preferably, the cancer which is treated is mesothelioma. Most preferably, the cancer which is treated is an asbestos-induced cancer.

The compositions according to the invention may have a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well tolerated by the subject to whom it is given.

Compositions according to the invention may be used in a number of ways. For instance, oral administration may be required, in which case the agents may be contained within a composition that may, for example, be ingested orally in the form of a tablet, capsule or liquid. Compositions comprising agents of the invention may be administered by inhalation (e.g. intranasally). Compositions may also be formulated for topical use. For instance, creams or ointments may be applied to the skin.

Agents or compositions according to the invention may also be incorporated within a slow- or delayed-release device. Such devices may, for example, be inserted on or under the skin, and the medicament may be released over weeks or even months. The device may be located at least adjacent to the treatment site. Such devices may be particularly advantageous when long-term treatment with agents used according to the invention is required and which would normally require frequent administration (e.g. at least daily injection).

In a preferred embodiment, compositions and agents according to the invention may be administered to a subject by injection into the blood stream or directly into a site requiring treatment. For example, the medicament may be injected at least adjacent to a sensitive cell, or within a tumour. Injections may be intravenous (bolus or infusion) or subcutaneous (bolus or infusion), or intradermal (bolus or infusion), or intrapleural.

It will be appreciated that the amount of the composition and agent that is required is determined by its biological activity and bioavailability, which in turn depends on the mode of administration, the physicochemical properties of the modulator and whether it is being used as a monotherapy or in a combined therapy. The frequency of administration will also be influenced by the half-life of the BAP1 inhibitors and DRLs within the subject being treated. Optimal dosages to be administered may be determined by those skilled in the art, and will vary with the particular agent in use, the strength of the pharmaceutical composition, the mode of administration, and the advancement of the cancer. Additional factors depending on the particular subject being treated will result in a need to adjust dosages, including subject age, weight, gender, diet, and time of administration.

Generally, a daily dose of between 0.01 μg/kg of body weight and 500 mg/kg of body weight of the agents (e.g. the inhibitor or the DRL) according to the invention may be used for treating, ameliorating, or preventing cancer, depending upon which agent is used. More preferably, the daily dose is between 0.01 mg/kg of body weight and 400 mg/kg of body weight, more preferably between 0.01 mg/kg and 200 mg/kg body weight, and most preferably between approximately 1 mg/kg and 100 mg/kg body weight.

The composition or agent(s) may be administered before, during or after onset of the cancer. Daily doses may be given as a single administration (e.g. a single daily injection). Alternatively, the agent may require administration twice or more times during a day. As an example, agents may be administered as two (or more depending upon the severity of the disease being treated) daily doses of between 25 mg and 7000 mg (i.e. assuming a body weight of 70 kg). A subject receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses.

Known procedures, such as those conventionally employed by the pharmaceutical industry (e.g. in vivo experimentation, clinical trials, etc.), may be used to form specific formulations comprising the agents according to the invention and precise therapeutic regimes (such as daily doses of the agents and the frequency of administration). The inventors believe that they are the first to describe a pharmaceutical composition for treating cancer, based on the use of a BAP1 inhibitor to sensitise the subject to death receptor-induced cell death, and a death receptor ligand, which induces cell death of the cells that express death receptors and previously insensitive to death receptor-induced cell death.

According to a tenth aspect, there is provided a pharmaceutical composition comprising the composition according to the sixth aspect, and a pharmaceutically acceptable vehicle.

According to an eleventh aspect, there is provided a method for preparing the pharmaceutical composition according to the tenth aspect, the method comprising contacting a therapeutically effective amount of a BAP1 inhibitor or agent that mimics the effect of BAP1 inhibition and a death receptor ligand, and a pharmaceutically acceptable vehicle.

A “therapeutically effective amount” of BAP1 inhibitor is any amount which, when administered to a subject, is the amount needed to sensitise an individual's cells to death receptor-induced cell death. A “therapeutically effective amount” of death receptor ligand is any amount which, when administered to a subject, is the amount needed to treat the cancer, or produce the desired effect.

For example, the therapeutically effective amount of active agent (i.e. BAP1 inhibitor and a death receptor ligand) used may be from about 0.01 mg/kg body weight to about 800 mg/kg body weight, and preferably from about 0.01 mg to about 500 mg. It is preferred that the amount of agent is an amount from about 0.1 mg to about 250 mg, and most preferably from about 0.1 mg/kg body weight to about 20 mg/kg body weight.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the peptide, antibody, DRL or BAP1 inhibitor) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidone, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions.

The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilised by, for example, intramuscular, intrathecal, epidural, intraperitoneal, intravenous and particularly subcutaneous injection. The composition or antibody may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agent or composition according to the invention can also be administered orally either in liquid or solid composition form. Compositions and agents suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “ variant” and “fragment”, can be a sequence that has at least 40% sequence identity with the amino acid/nucleotide/peptide sequences of any one of the sequences referred to herein, for example 40% identity with the polypeptide identified as SEQ ID Nos. 3 or 4, and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value.

The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix =Gonnet. For DNA and Protein alignments: ENDGAP=−1, and GAPDIST=4. Those skilled in the art will be aware that it may be necessary to vary these and other parameters for optimal sequence alignment. Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to any sequences referred to herein or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID Nos. 3 or 4.

Due to the degeneracy of the genetic code, it is clear that any nucleic acid sequence described herein could be varied or changed without substantially affecting the sequence of the protein encoded thereby, to provide a variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

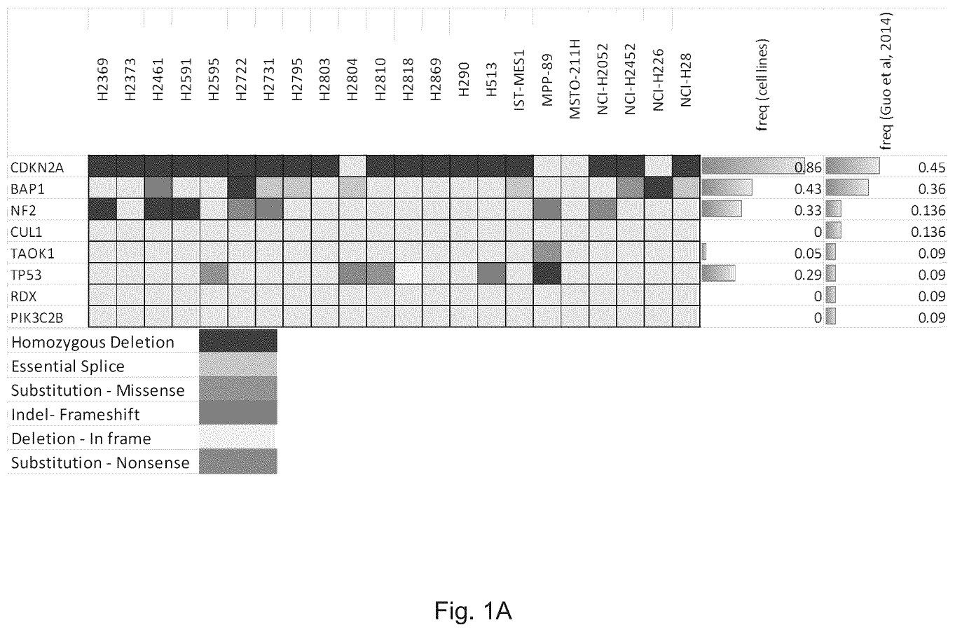

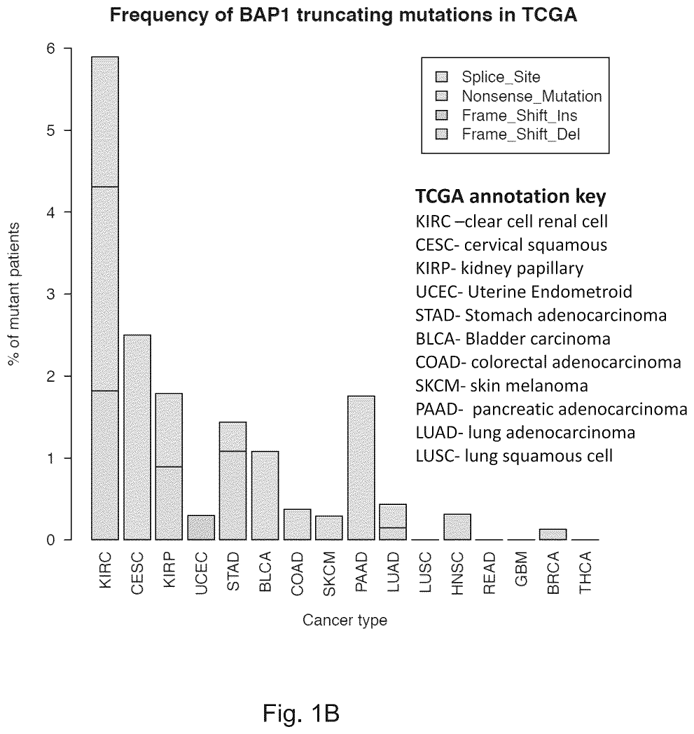

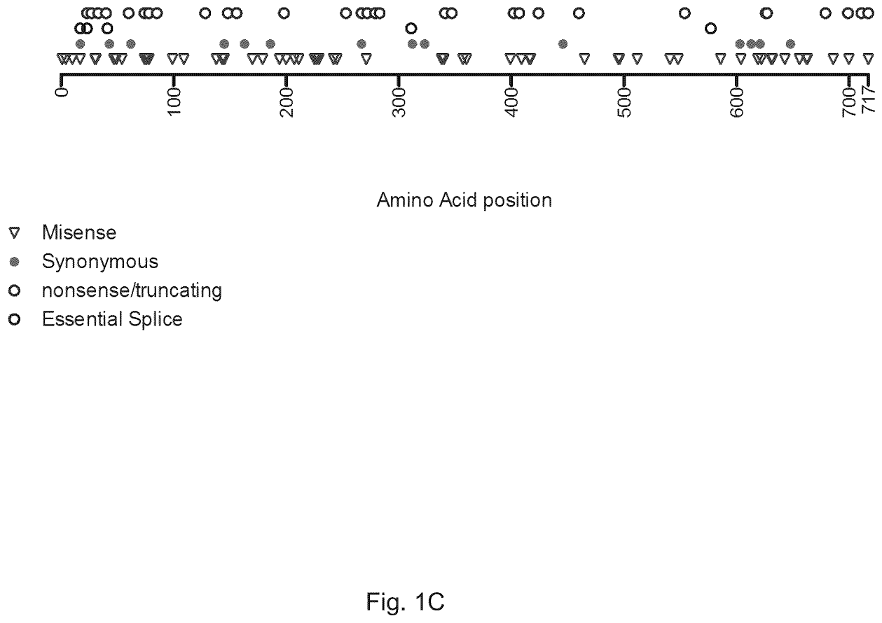

FIG. 1A shows various types of mutations in different malignant pleural mesothelioma (MPM) cell lines. FIG. 1B is an analysis of TCGA exomes for enrichment of BAP1 loss of function mutations. FIG. 1C is a schematic of BAP1 gene exons with Mutations annotated from 5180 TCGA exomes.

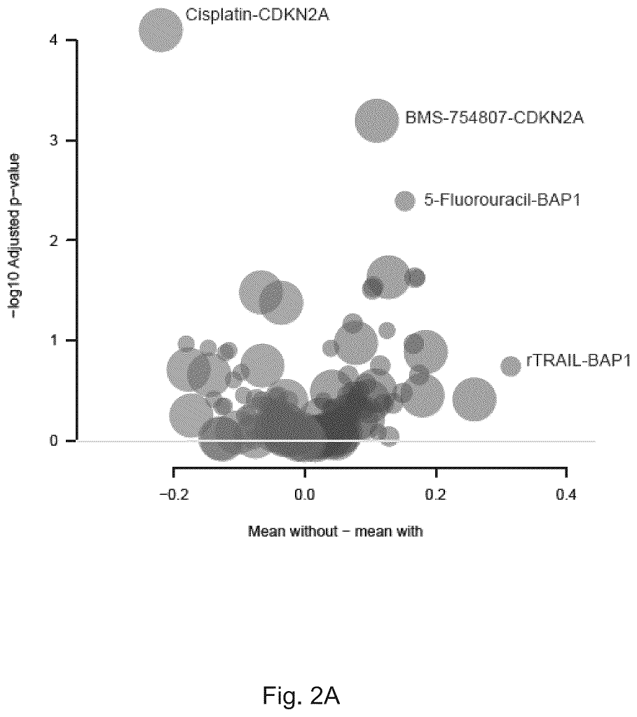

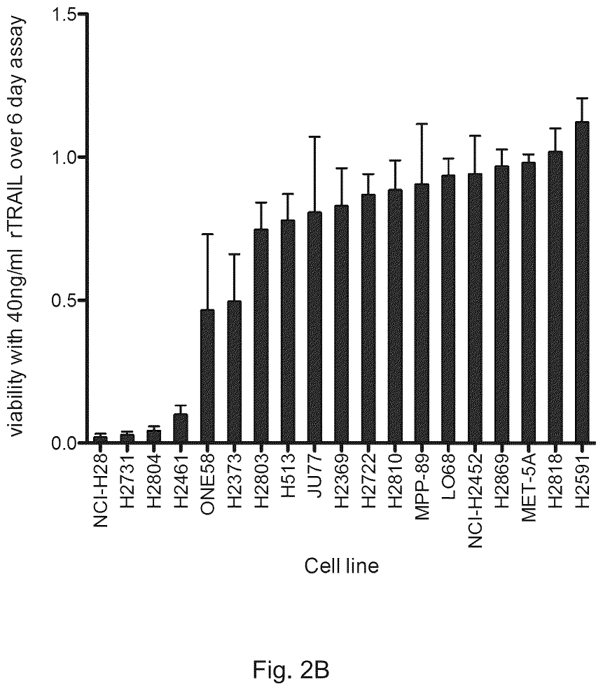

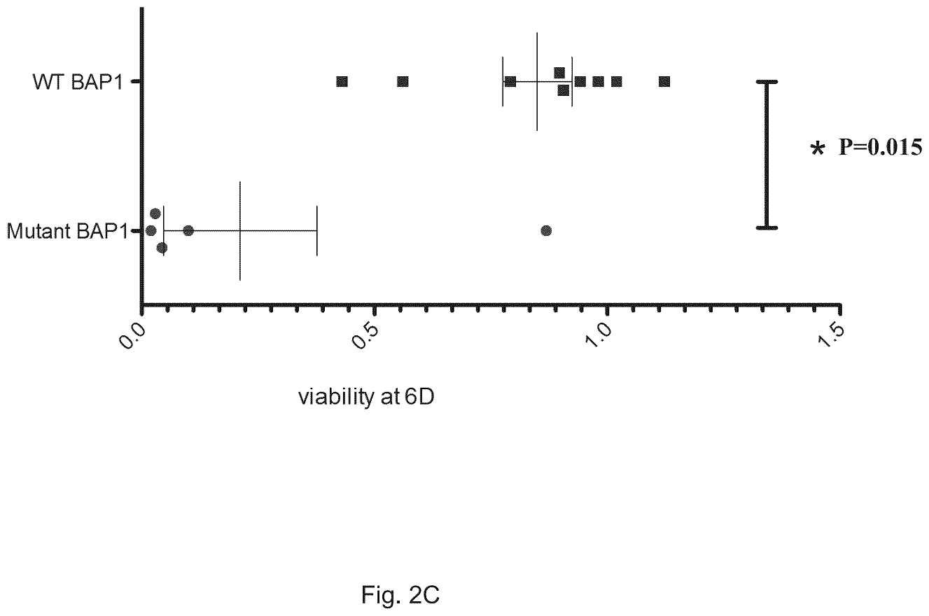

FIG. 2A is a volcano plot showing drug-genome interactions in MPM. The volcano plot displays the mean delta AUC by genotype for 92 library compounds. The Y-axis denotes adjusted p-Value, X-axis denotes effect size. Size of circle indicates number of mutant lines in cohort tested. FIG. 2B is a histogram which shows the results of a 6-day viability assay for multiple mesothelioma cell lines (n=19). rTRAIL (40 ng/ml). Met 5a is a mesothelial normal control line. FIG. 2C is a scatter plot showing AUC following 6 day viability assay in response to 40 ng/ml rTRAIL (normalised to DMSO treated control) in cell lines discretized by BAP1 mutation status. BAP1 mutation status significantly correlates with response to rTRAIL. Two-tailed t-test, p=0.015.

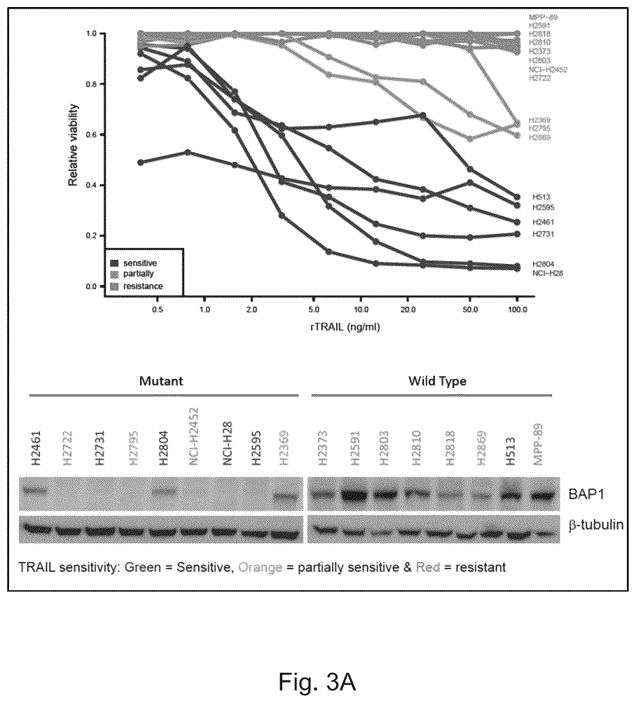

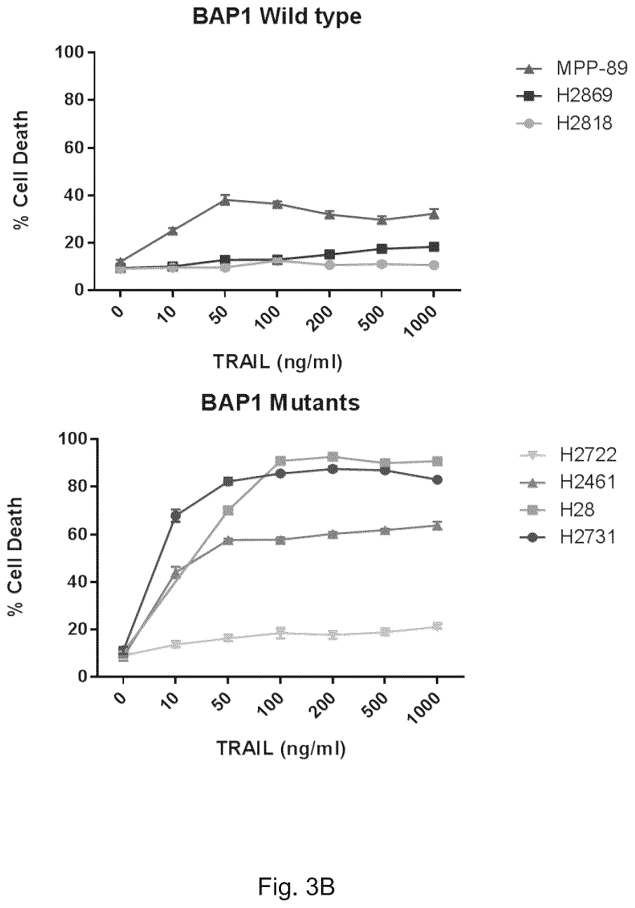

FIG. 3A shows the results of a viability assay in which various MPM cells lines are treated with rTRAIL. MPM cell lines were treated with a dose range from 0.5 ng/ml to 100 ng/ml and cell viability was measured using Syto-60 assay. Based on their cell viability, the cell lines were classified into resistant (red), partially sensitive (orange) and sensitive (green). Cell lines were western blotted to probe the expression of BAP1 protein expression. FIG. 3B shows the results of a viability assay in which various MPM cells lines are treated with rTRAIL. Three BAP1 wild-type cell lines (MPP-89, H2869 & H2818) and four BAP1 mutant cell lines (H2722, H2461, H28 and H2731) were treated with TRAIL (0-1000 ng/ml) for 24 hours and cell death quantified using an Annexin V/DAPI cell death assay.

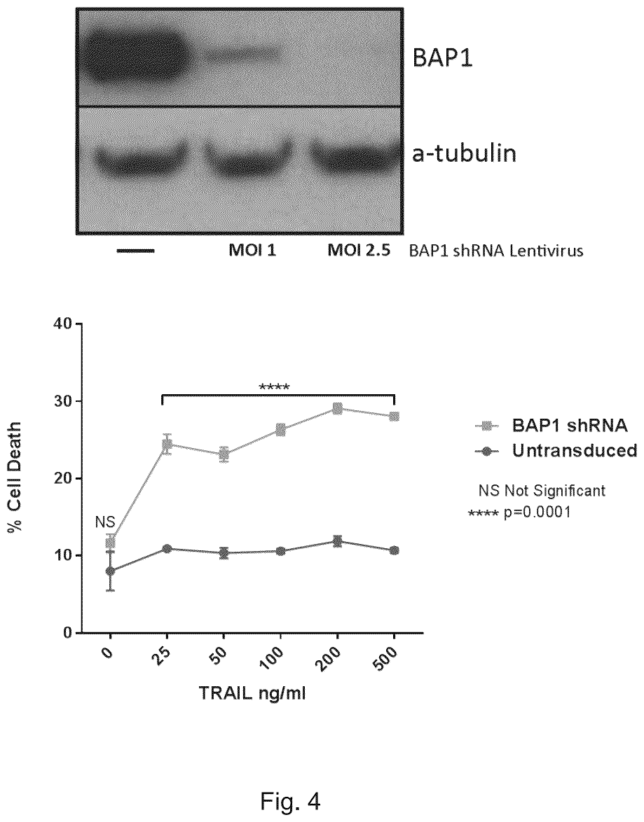

FIG. 4 is a graph (and Western blot), which shows that knocking down BAP1 in a BAP1 wild-type mesothelioma cell line H2818 confers increased cell death response to rTRAIL.

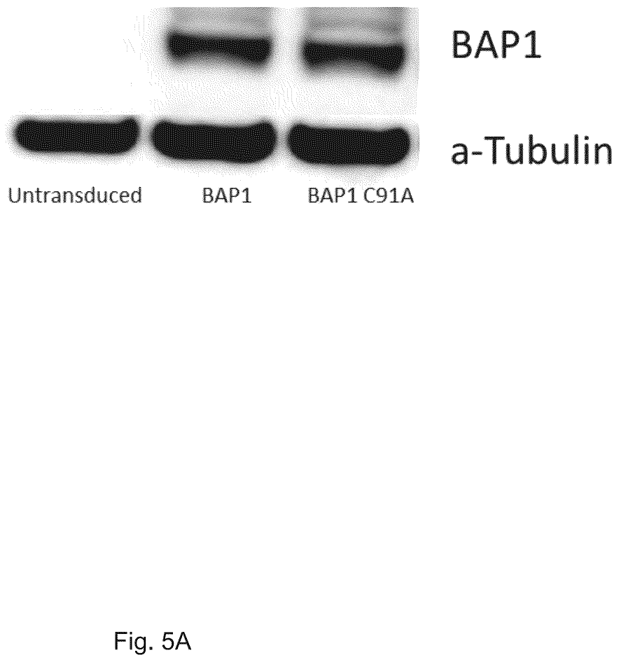

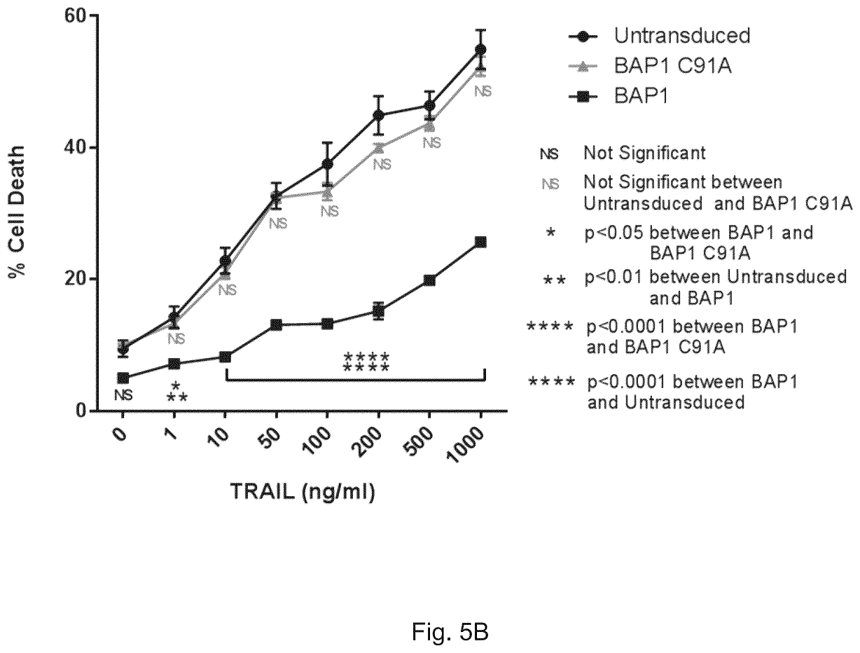

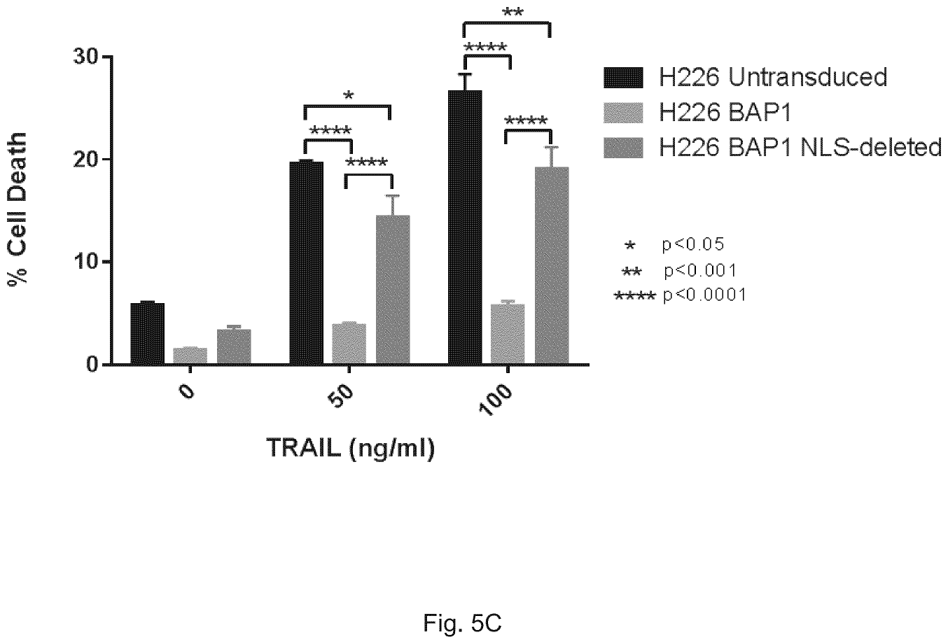

FIG. 5A is an immunoblot for BAP1 protein in BAP1 null mesothelioma lines following transfection with empty vector and BAP1 expression vector. FIG. 5B is a dose-response curve for an Annexin V/DAPI cell death assay performed with rTRAIL on the BAP1 null H226 parental line, a BAP1 wt overexpressing stable line, and BAP1 c91 hydrolase inactive stable cell line. FIG. 5C is a graph showing the effect of rTRAIL on cell viability of an untransduced H226 cell line, a BAP1 (transduced) cell line, and a H226 cell line with the NLS deleted.

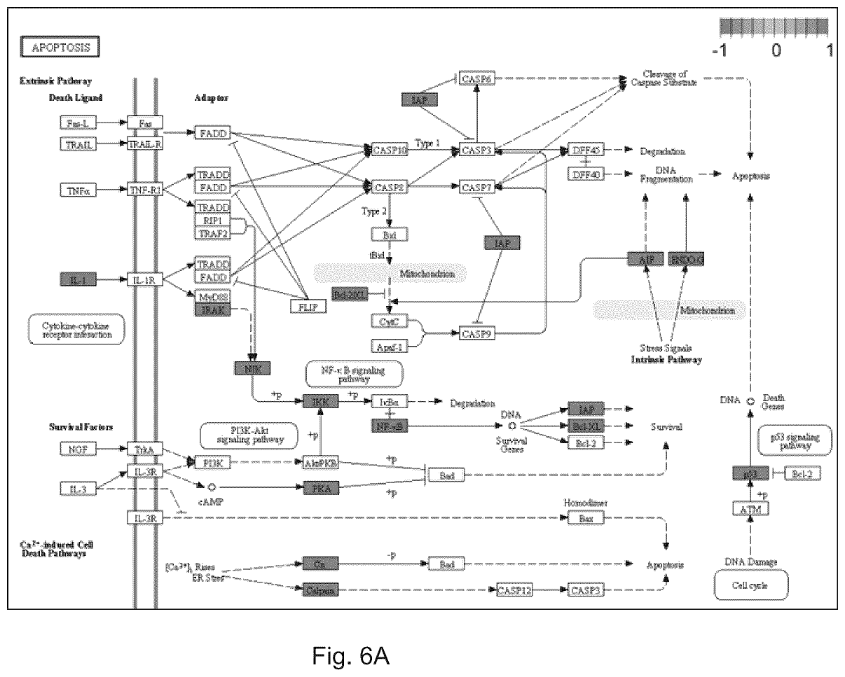

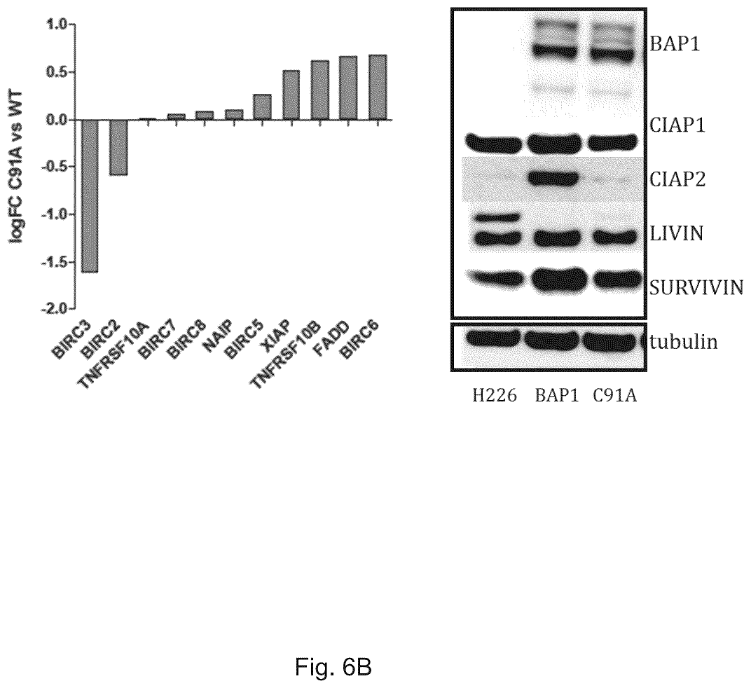

FIG. 6A shows that cell death is significantly dysregulated with the loss of the BAP1 catalytic ubiquitin hydrolation domain. Comparing GEX profile of C91 variant (catalytically inactive) BAP1 transduced H226 with H226 BAP1 wild-type transduced H226 Kegg pathway analysis on significantly dysregulated genes analysis with adj p <0.05 and FDR <20%. FIG. 6B is a graph showing RMA normalised gene centered mRNA expression of IAP genes BIRC2 and BIRC3 in c91 mutant BAP1 vs wild-type BAP1 expressing H226 cell line. The Western blot shows dysregulation of IAP family proteins in H226 cell line expressing catalytically inactivated C91 mutant BAP1.

FIG. 7A is a volcano plot showing drug-genome interactions when rTRAIL was used as an anchor drug in combination with the library of 94 single agent compounds. Synergy was described using delta AUC metric. FIGS. 7B & 7C are graphs showing the effect of rTRAIL on cell viability of MPM cells in the presence of LCL161. TRAIL resistant MPM cells were treated with either 0-1000 ng/ml of TRAIL alone or a combination of 5 μM LCL161 and 1-1000 ng/ml of TRAIL for 24 hours and cell death was quantified by Annexin V/DAPI assay. FIG. 7D is a graph showing the effect of rTRAIL on cell viability of cells in the presence of the IAP inhibitor, LCL161. BAP1-transduced or BAP1 C91A-transduced H226 cells were treated either with 0-1000 ng/ml of TRAIL alone or combination of 5 μM LCL161 and 0-1000 ng/ml of TRAIL for 24 hours and cell death was quantified by Annexin V/DAPI assay.

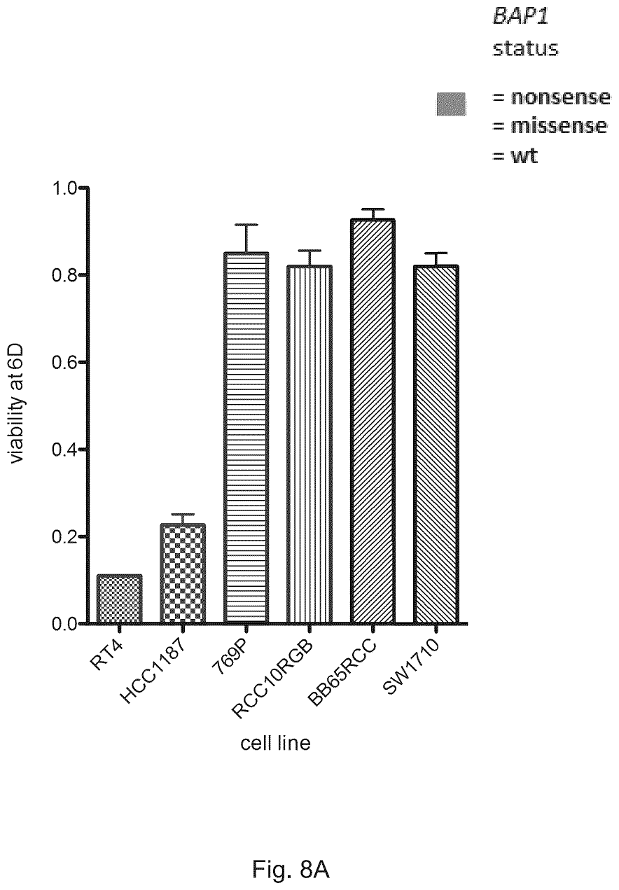

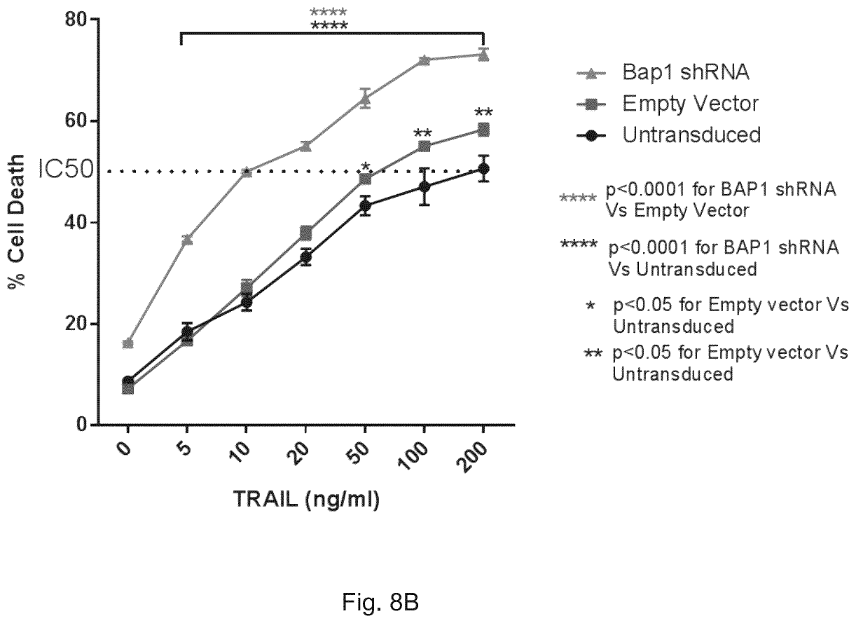

FIG. 8A is a graph showing the effect of rTRAIL on the viability of various cancer cell lines. Bladder (RT4) and Breast (HCC1187) cancer cell lines with nonsense mutations in BAP1 show sensitivity to rTRAIL while renal cell cancer cell lines (769P & RCC10RGB) with missense mutation and wild-type renal (BB65RCC) and bladder cancer (SW1710) cell lines are resistant to TRAIL. FIG. 8B shows that knockdown of BAP1 in Breast cancer cell line MDA MB-231 increases sensitivity to rTRAIL.

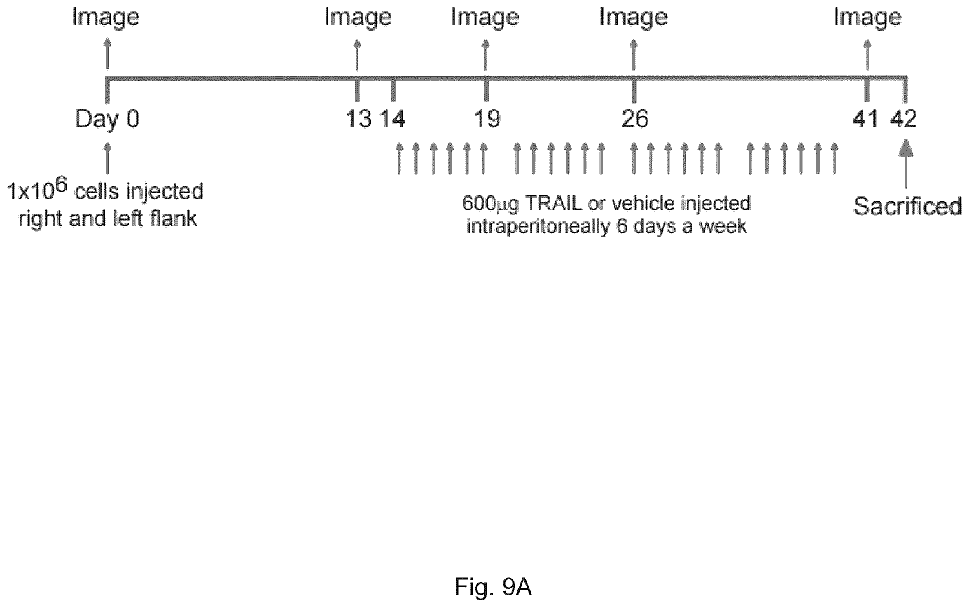

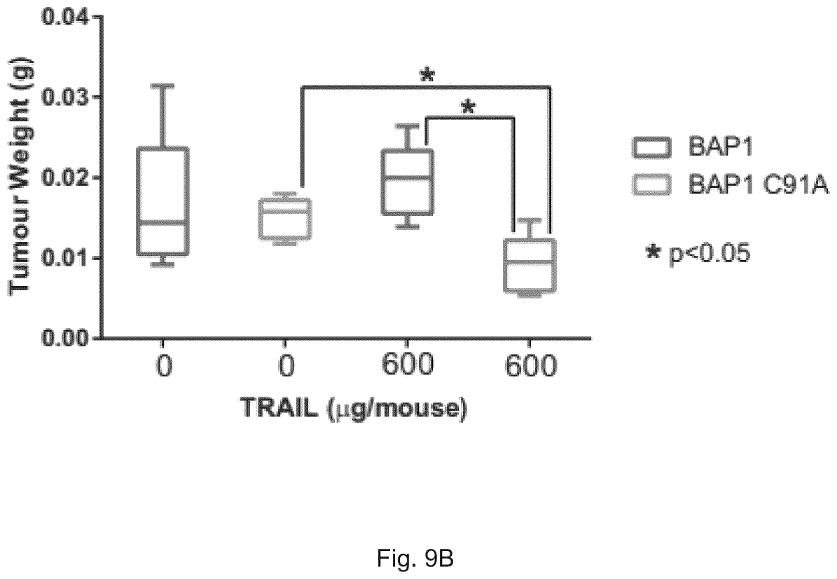

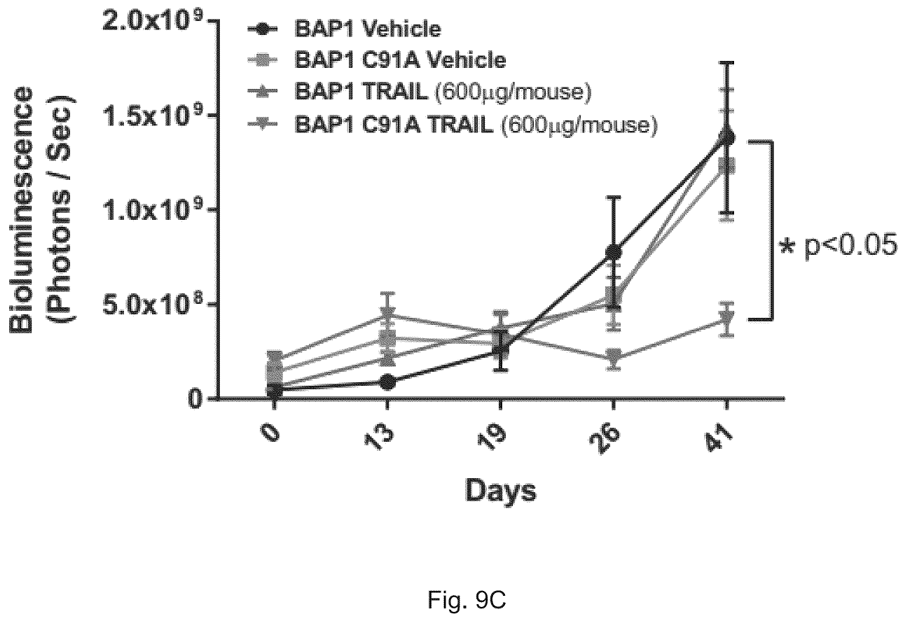

FIG. 9A is a protocol of an in vivo experiment. FIG. 9B is a box plot showing the weight of tumours extracted from mice injected with mutant or wild-type BAP1-expressing cells after treatment with rTRAIL. Tumour weights of mutated BAP1 xenografts are significantly smaller than wild-type BAP1 xenografts after TRAIL treatment. FIG. 9C is a graph showing that TRAIL treatment reduces the tumour burden (measured by bioluminescence) of mutated BAP1 xenografts when compared to TRAIL treated wild-type BAP1 xenografts or untreated BAP1 mutated and BAP1 wild-type xenografts.

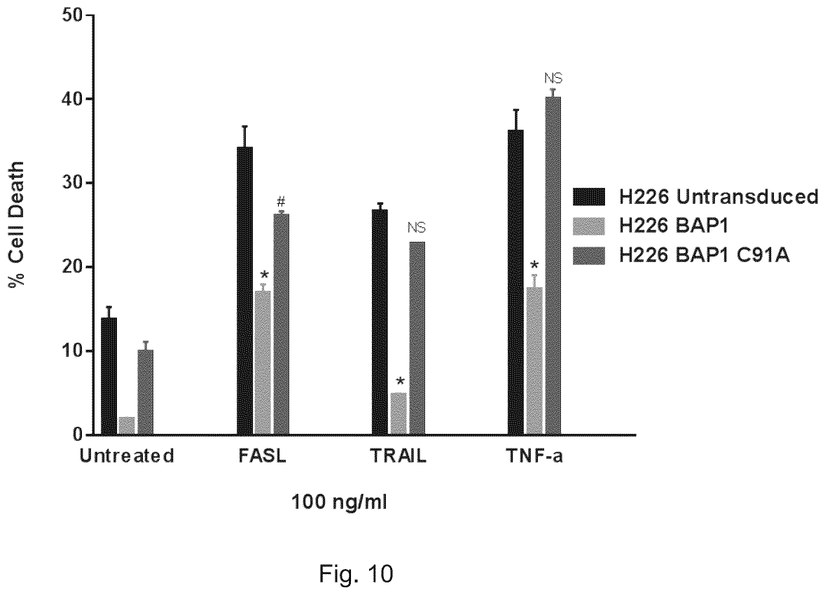

FIG. 10 is a graph, which shows that BAP1 the that catalytic domain of BAP1 also regulates the sensitivity of H226 cells to the cell death-inducing ligands, TRAIL, FASL and TNFα. Untransduced BAP1-negative H226 cells, BAP1-expressing and catalytically dead BAP1-expressing H226 cells were treated with too ng/ml of FASL, TRAIL and TNF-alpha for 24 hours and cell death was quantified by Annexin V/DAPI assay. *p<0.05 indicating significant difference between untransduced H226 cells H226 BAP1 expressing cells. NS no significant difference between untransduced H226 cells and BAP1 C91A transduced cells. #p<0.05 indicating significant difference between untransduced H226 cells and BAP1 C91A transduced cells.

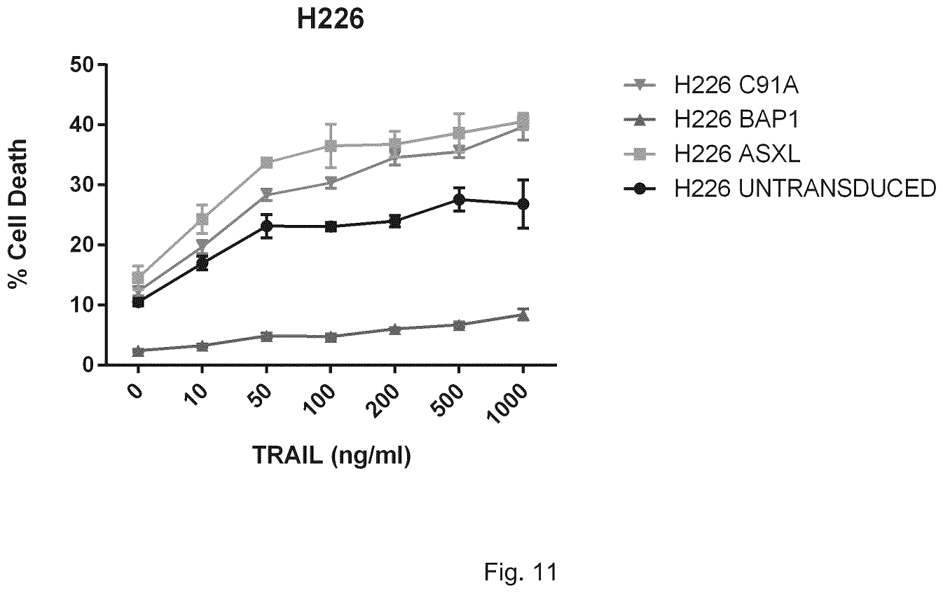

FIG. 11 is a graph, which shows that BAP1 that catalytic domain of BAP1 also regulates the sensitivity of H226 cells to the cell death inducing ligands, TRAIL, FASL and TNFα. Untransduced BAP1-negative H226 cells, BAP1-expressing, ASXL binding site deleted BAP1-expressing H226 cells and catalytically dead BAP1-expressing H226 cells were treated with 100 ng/ml of FASL, TRAIL and TNF-alpha for 24 hours and cell death was quantified by Annexin V/DAPI assay.

### EXAMPLES

The inventors have discovered that mutation of the BAP1 tumour suppressor gene confers sensitivity to therapeutic modulation of the apoptotic pathway in human cancers. They have explored and validated this association in malignant pleural mesothelioma, bladder carcinoma and breast carcinoma and have evidence that it can be extended to between 1-36% human cancers, including renal cell carcinoma, and cervical cancer and uveal melanoma. Although the data described herein focuses on rTRAIL, a recombinant protein that activates the TRAIL pathway by binding to TRAIL receptor 1 (TRAIL-R1, also known as death receptor 4; DR4) and TRAIL-R2 (also known as DR5), BAP1 is also found to modulate other pro-apoptotic pathways, such as the FAS ligand pathway or the TNF pathway or intrinsic apoptotic pathway (see Example 6).

### MATERIALS AND METHODS

### Whole Exome Sequencing

DNA was extracted using the column extraction technique as per manufacturer's instructions (QIAGEN). Genomic libraries were prepared using the Illumina paired end sample prep kit following the manufacturer's instructions. Exome enrichment was performed using the Agilent SureSelect Human All Exon 50 Mb kit following the manufacturer's recommended protocol. Each exome was sequenced using the 75-bp paired end protocol on an Illumina HiSeq 2000 DNA Analyser to produce approximately 5-10 Gb of sequence per exome. Sequencing reads were aligned to the human genome (NCBI build GrCh 37) using the Burrows-Wheeler aligner (BWA) algorithm with default settings (17). Unmapped reads and PCR duplicates were excluded from the analysis. Average coverage of the cell line exomes at 20× or higher was 80%.

### Copy Number Annotation

DNA was extracted as above. DNA was outsourced to AROS for SNP 6.o array (http://arosab/services/microarrays/genotyping/). Copy number annotation was derived from the PICNIC algorithm [18].

### Variant Detection

The CaVEMan algorithm was used to call single nucleotide substitutions [19]. The algorithm uses a naïve Bayesian classifier to estimate the posterior probability of each possible genotype (wild-type, germline or somatic mutation) at each base. To call insertions and deletions, split read mapping was implemented as a modification of the Pindel Algorithm [19]. Pindel searches for one read anchored on the genome with the other read mapped in two portions, spanning a putative insertion/deletion. For both algorithms, an identical putative normal from the CGP panel of tumours was nominated that has been used in all cell lines studied without available matched normal tissue. Significant post processing filtering against various panels of normal was subsequently undertaken to eliminate as many germline single nucleotide polymorphisms as possible. These include the 1000 genomes database, DB SNP, and an internal panel of CGP normal. Following these steps missense variants were annotated using the FATHM algorithm (Cancer Genome Project) as to potential functional consequence of the variant.

### Combination (Genome-Drug) Therapeutic Screen Approaches

Manual “single dose” combination screening was undertaken using 96 well formats. Cells were plated on day 1 in previously optimized seeding densities in 180 μl if media. On day 2 20 μl of a 10× concentration of media from a stock of drugs was added. Cells were then allowed to grow for 72 hrs or 6 days and fixed at the end of the assay. Drug wells were compared to DMSO treated control wells.

Single agent high throughput 5 point viability screening was undertaken in 384 well formats using robotic liquid handling with fixing with 4% paraformaldehyde and staining for viability with Syto60 nucleic acid dye (Invitrogen) (see below). Single agent dose response curves were derived for each library of 85-95 drugs according to the experiment used, and log IC50 or area under the curve (AUC) metrics were derived for each library compound in each cell line according to a previously derived formula [18]. Using this data various 2-drug synergy was measured with a Delta AUC metric.

A binary event matrix was compiled for the cell lines in the mesothelioma screen by aggregating copy number and exome data and this was used as input classifiers for genomic correlation. Data from this therapeutic screen was then analysed using a Multivariate Analysis of Variance (MANOVA) [18] to annotate the sensitizing effect of genotype on dose response. The results are presented as a volcano plot demonstrating significance of the interaction (above a Benjamin Hochberg false discovery threshold) and magnitude of effect size.

### Analyses of TCGA Data

Frequency of BAP1 truncating mutations in various cancer types is based upon data generated by The Cancer Genome Atlas (TCGA) Research Network: http://cancergenome.nih.gov/.

### Cell Culture

293T cells were cultured in Dulbecco's modified Eagle's medium (DMEM) supplemented with 10% fetal bovine serum (FBS), nonessential amino acids, 50 U/mL penicillin, 50 μg/mL streptomycin, and 1% sodium pyruvate. Human mesothelioma cell lines were cultured in RPMI-1640 medium supplemented with 10% FBS, 1% penicillin-streptomycin and 1% sodium pyruvate (H2369, H2373, H2461, H2591, H2595, H2722, H2731, H2795, H2803, H2804, H2869, H290, H5 1 3, IST-MES1, MPP-89, MSTO-211H, NCI-H2052, NCI-H2452, NCI-H226, NCI-H28) or Dulbecco's Modified Eagle Medium/Nutrient Mixture F-12 (DMEM/F-12) supplemented with 10% FBS, nonessential amino acids, 50 U/mL penicillin, 50 μg/mL streptomycin, and 1% sodium pyruvate (H2818, H2810). Cells were maintained at 37° C. at 5% CO₂.

### Western Blotting

Cell monolayers were washed in phosphate buffered saline (PBS) and lysed in radioimmunoprecipitation assay (RIPA) buffer (Sigma-Aldrich) and protease inhibitors (Complete-mini; Roche) on ice. Lysates were centrifuged at 14000 rpm for ten minutes and the supernatant aspirated. Protein concentration was calculated from a standard curve of bovine serum albumin using the BCA assay (Calbiotech) according to the manufacturer's instructions. Lysates were prepared to the appropriate concentration and 4× Laemelli buffer and 10× reducing agent added prior to the sample being heated at 70° C. for ten minutes. Lysates were subjected to SDS-PAGE on pre-cast 4-12% Bis-Tris gels (Invitrogen) at 200V for 1 hr. Protein was transferred onto a nitrocellulose membrane using an iBlot gel transfer device (Invitrogen) as per manufacturer's instructions. Membranes were blocked in 5% milk in tris-buffered saline with Tween 20 (TBS-T) before the addition of primary antibody (at 1:1000 in TBS-T unless otherwise stated) overnight at 4° C. The following day the membrane was washed three times in TBS-T and secondary antibody added (at 1:2500 in TBS-T) Antibodies used include BAP1 (C-4; Santa Cruz sc-28383), alpha tubulin (Cell Signalling #2125), c-IAP1 (Cell signaling #7065), cIAP2 (Cell signaling, #3130), Livin (Cell Signalling #5471), Survivin (Cell signaling, #2803), Alexa Fluor® 488 (Invitrogen A-21202). Immunoblots were imaged using an ImageQuant™ LAS 4000 biomolecular imager.

### Cell Viability Assays

Adherent cell lines were seeded 24 hours before drugging. Cells were trypsinised and counted before seeding at the optimal density for the size of well (96 or 384) and duration of assay. 72 hr after drug treatment cells were fixed with 4% paraformaldehyde for 30 minutes. Following two washes of dH2O 100 μl Syto60 nucleic acid stain (Invitrogen) was added at a final concentration of 1 μM and plates fixed for 1 hr at room temperature. Quantification of fluorescent signal was achieved using excitation/emission wavelength of 630/695 nM.

### Cell Death Assays

Adherent mesothelioma cell lines were plated in a 96 well plate at approximately 10000 cells per well. Cells were plated and given 1 day to adhere at which time drug was added. After 48 hrs media, including floating cells, was collected from each well. The remaining adherent cells were washed with PBS and mobilised with 0.05% trypsin in EDTA. All cells were collected into tubes containing the previously removed media and pelleted by centrifugation (300 g, 5 minutes). Cells were then re-suspended in ix Annexin V binding buffer with 10 μl/1 ml concentration of Annexin V-647 antibody (Invitrogen) and incubated for 15 minutes at room temperature. DAPI (2 μg/ml) was then added to each sample before flow cytometry analysis as below. Annexin V−/DAPI− cells were judged to be viable, AnnexinV+/DAPI− cells were considered to be undergoing apoptosis (early apoptotic phase), and Annexin V+/DAPI+ cells were considered late apoptotic or necrotic, and recorded as dead.

### Flow Cytometry Analyses

Cells were washed with phosphate buffered saline and fixed by incubation in 4% paraformaldehyde for 20 minutes at room temperature. For intracellular BAP1 staining, fixed cells were permeabilised in 0.1% triton X-100 in PBS for 20 minutes on ice, washed twice with PBS, incubated with primary antibody (C-4; Santa Cruz sc-28383, 1:100) for 20 minutes on ice, washed twice again and incubated with a fluorescent secondary antibody (Alexa Fluor® 488, 1:200 (Invitrogen A-21202)). Cells were washed twice with PBS and suspended in PBS for flow cytometry analysis. Cells analysed as part of the cell death assays were prepared as above.

Flow cytometry analysis was conducted on an LSRFortessa cell analyser (BD Biosciences) and data analysed with FlowJo software.

mRNA Microarray

The mRNA from catalytically inactive BAP1 expressing H226 cells (H226 C91A) and WT BAP1 expressing H226 cells (H226 BAP1) was extracted and run on an Illumina HT12 array.

### Pathway Analysis

The significantly differentially expressed genes identified from the mRNA microarray were analysed using KEGG pathway analysis.

### Plasmids

The cDNA full-length clone of human BAP1 was obtained in a pCMV6-AC backbone (Origene, SC117256), which was cloned into a PCCL.CMV lentiviral backbone for all further experiments. BAP1 mutant constructs were generated using site directed mutagenesis kits (NEB) and confirmed by full length DNA sequencing. Short hairpin RNAs were obtained through UCL RNAi library in a GIPZ shRNAmir lentiviral vector (Dharmacon V2LHS41473). The sequence (SEQ ID NO. 44) for the short hairpin is as follows:

[SEQ ID NO. 44]

TAAAGGTGCAGATGAACTC

### Lentivirus Production and Concentration

Lentiviruses were generated by transfection of 293T cells with the lentivirus vector plasmids together with the packaging plasmid pCMVdR8.2 and envelope plasmid pMDG.2 using jetPEI (Polyplus Transfection) as the transfection reagent. The 293T cells were incubated at 37° C. and the medium containing the lentiviruses harvested at 24 and 48 hrs. The lentivirus was concentrated by ultracentrifugation at 18000 rpm for 2hours at 4° C. (SW28 rotor, Optima LE80K Ultracentrifuge, Beckman) and stored at −80° C. before use.

Lentivirus titration was performed by transducing 293T cells with serial dilutions of virus in the presence of 4 μg/ml polybrene. After 4 days cells were analyzed for the percentage of BAP1 positive cells using flow cytometry. Viral was calculated as follows:

Titre (transduction units (TU)/ml)=Proportion of BAP1 positive cells×number of seeded cells/volume of virus (ml)

MPM cells were then transduced with a range of multiplicity of infections (MOIs) in the presence of 4 μg/ml polybrene and transduction efficacy assessed by flow cytometry analysis. The optimal population (lowest MOI at which >90% transduction achieved) was selected for further experiments.

shRNA Experiments

Lentivirus encoding shRNA targeting BAP1 was generated as per the lentivirus production protocol above. MPM cells (H2818) were transduced and treated with puromycin 200 μg/mL until a pure population was achieved. Immunoblotting was performed to assess efficacy of the shRNA knockdown.

### Animals

All animal studies were approved by the University College London Biological Services Ethical Review Committee and licensed under the UK Home Office regulations and the Evidence for the Operation of Animals (Scientific Procedures) Act 1986 (Home Office, London, UK). Mice were purchased from Charles River, kept in individually ventilated cages under specific pathogen-free conditions and had access to sterile-irradiated food and autoclaved water ad libitum.

### Xenograft Mouse Models

Groups of 8 week old NOD.CB17-Prkdcscid/NcrCrl (NOD SCID) mice (Charles River) were injected on each flank with 1 million cells of luciferase transduced mesothelioma cell lines (H226 BAP and H226 C91A) in a 1:1 mixture of matrigel and media. When tumours were established, as assessed by bioluminescent imaging (IVIS), at 14 days following injection of tumour cells, treatment was began with either vehicle or isoleucine zipper TRAIL (izTRAIL) [20]. Either vehicle or izTRAIL were given

intraperitoneally once daily at a dose of 600 mcg for the duration of the experiment. Tumour size was assessed at days 0, 13, 19, 26 and 41 using bioluminescent imaging (IVIS). Mice were culled at 42 days and tumours removed and weighed. Six mice per group were treated. Researchers were not blinded in these experiments.

### Statistical Analyses

Statistical analysis was performed using GraphPad Prism V.4 (GraphPad Software). In vivo experiments with multiple groups were analysed using repeated measures ANOVA, and single-group data were assessed using Student t test. All in vitro experiments were performed in triplicate unless specified otherwise.

### Example 1

### TRAIL Targets BAP1 Mutant Mesothelioma Cells

The inventors carried out a combinatorial chemical screen in 15 mesothelioma cell lines (together with the Met5a mesothelial normal control line) using 94 small molecule inhibitors and chemotherapy agents (see Table 1) either alone or in combination with the ligand tumour necrosis factor (TNF)-related cell death inducing ligand (TRAIL). To detect examples of extreme drug sensitivity, the inventors analysed for statistical associations between response and the mutational status of these cell lines based on a set of 8 genes recently identified as being candidate cancer genes in mesothelioma (see FIG. 1A). Of note BAP1 mutations are well recognised across cancer types (see FIGS. 1B and C). The largest effect of a mutation on drug response was that of mesothelioma cells harbouring a mutation in the deubiquitinase BAP1 and treated with TRAIL (see FIG. 2A). There was no significant effect on cell viability observed in the control normal mesothelial cell line MET-5A included in the screen (see FIG. 2B). BAP1 mutant cells were significantly more sensitive to TRAIL than their wild-type counterparts (see FIGS. 2B & 2C). Furthermore, the BAP1 mutations detected in these cell lines would be predicted to be truncating (see Table 2). The inventors confirmed by immunoblot that BAP1 mutations were usually associated with loss of protein expression and the mutant cell lines are generally sensitive to TRAIL (see FIG. 3A).

TABLE 1
Compounds used in combinatorial chemical screen with 15 mesothelioma
cell lines together with the Met5a mesothelial normal control line.

| | | | min | max |
|---|---|---|---|---|
| | | Targeted | conc | conc |
| compound | target | process/pathway | (uM) | (uM) |
| AICAR | AMPK agonist | metabolism | 7.81 | 2000 |
| Camptothecin | DNA topoisomerase I | DNA replication | 0.0004 | 0.1 |
| Vinblastine | Microtubules | cytoskeleton | 0.0004 | 0.1 |
| Cisplatin | DNA crosslinking | DNA replication | 0.0234 | 6 |
| Docetaxel | Microtubules | cytoskeleton | 0.0000 | 0.0125 |
| Gefitinib | EGFR | EGFR signalling | 0.0020 | 0.5 |
| ABT-263 | Bcl-2, Bcl-xL, and Bcl-w | apoptosis regulation | 0.0078 | 2 |
| Vorinostat | HDAC inhibitor Class I, IIa, | chromain histone | 0.0391 | 10 |
| | IIb, IV | acetylation | | |
| Nilotinib | Bcr-Abl | ABL signalling | 0.0078 | 2 |
| AZD-2281 | PARP1/2 | Genome integrity | 0.0195 | 5 |
| Bosutinib | SRC, ABL, TEC | ABL signalling | 0.0078 | 2 |
| Lenalidomide | TNF alpha | other | 0.0195 | 5 |
| Axitinib | PDGFR, KIT, VEGFR | RTK signalling | 0.0078 | 2 |
| AZD7762 | Chk 1/2 | Genome integrity | 0.0078 | 2 |
| GW 441756 | Trk A | RTK signalling | 0.0078 | 2 |
| CEP-701 | FLT3, JAK2, NTRK1, RET | RTK signalling | 0.0078 | 2 |
| SB 216763 | GSKa/b | WNT signalling | 0.0391 | 10 |
| 17-AAG | Hsp90 | other | 0.0039 | 1 |
| AMG-706 | VEGFR, RET, c-KIT, PDGFR | RTK signalling | 0.0078 | 2 |
| KU-55933 | ATM | Genome integrity | 0.0391 | 10 |
| BIBW2992 | EGFR, HER2 | EGFR signalling | 0.0020 | 0.5 |
| GDC-0449 | SMO | other | 0.0391 | 10 |
| PLX4720 | RAF | ERK MAPK | 0.0391 | 10 |
| | | signalling | | |
| BX-795 | TBK1, PDK1, IKK, AURKB/C | other | 0.0195 | 5 |
| NU-7441 | DNAPK | Genome integrity | 0.0078 | 2 |
| SL 0101-1 | RSK, AURKB, PIM3 | ERK MAPK | 0.0391 | 10 |
| | | signalling | | |
| BI-D1870 | RSK1/2/3/5, PLK1, AURKB | cell cycle | 0.0195 | 5 |
| BIRB 0796 | p38, JNK2 | JNK and p38 | 0.0391 | 10 |
| | | signalling | | |
| JNK Inhibitor VIII | JNK | JNK and p38 | 0.0391 | 10 |
| | | signalling | | |
| 681640 | Wee1, Chk1 | cell cycle | 0.0078 | 2 |
| Nutlin-3a | p53-MDM2 interaction | p53 pathway | 0.0313 | 8 |
| mirin | MRE11-Rad50-Nbs1 complex | cell cycle | 0.3906 | 100 |
| PD-173074 | FGFR1, FGFR3 | RTK signalling | 0.0078 | 2 |
| ZM-447439 | Aurora B | mitosis | 0.0156 | 4 |
| RO-3306 | Cdk1 | cell cycle | 0.0195 | 5 |
| MK-2206 | AKT1/2 | PI3K signalling | 0.0156 | 4 |
| PD-0332991 | Cdk 4/6 | cell cycle | 0.0156 | 4 |
| PF477736 | Chk 1 (Chk2) | cell cycle | 0.0039 | 1 |
| GW843682X (AN-13) | Plk1 | mitosis | 0.0195 | 5 |
| NVP-BEZ235 | PI3K Class 1 and mTORC1/2 | PI3K signalling | 0.0010 | 0.25 |
| GDC0941 | PI3K (class 1) | PI3K signalling | 0.0156 | 4 |
| AZD8055 | mTORC1/2 | TOR signalling | 0.0078 | 2 |
| PD-0325901 | MEK 1/2 | ERK MAPK | 0.0010 | 0.25 |
| | | signalling | | |
| AZD6482 | PI3K beta | PI3K signalling | 0.0195 | 5 |
| Obatoclax Mesylate | Bcl-2, Bxl-xl, Mcl-1 | apoptosis regulation | 0.0391 | 10 |
| EHT 1864 | Rac GTPases | cytoskeleton | 0.0391 | 10 |
| BMS-708163 | gamma-secretase complex | other | 0.0195 | 5 |
| 5-Fluorouracil | antimetabolite | mitosis | 0.0781 | 20 |
| Paclitaxel | Beta subunit of tubulin | cytoskeleton | 0.0000 | 0.01 |
| PF-02341066 | MET, ALK | RTK signalling | 0.0039 | 1 |
| Sorafenib | PDGFR, KIT, VEGFR | RTK signalling | 0.0156 | 4 |
| BI-2536 | Plk1, 2, 3 | mitosis | 0.0020 | 0.5 |
| BMS-536924 | IGF-1R | IGFR signalling | 0.0156 | 4 |
| GSK1904529A | IGF-IR and IR | IGFR signalling | 0.0195 | 5 |
| AKT inhibitor VIII | AKT1/2/3 | PI3K signalling | 0.0195 | 5 |
| PF-4708671 | p70 S6KA | TOR signalling | 0.0391 | 10 |
| JNJ-26854165 | MDM2 | p53 pathway | 0.0391 | 10 |
| LY317615 | PKC beta | other | 0.0391 | 10 |
| BMS-754807 | IGF-1R/IR | IGFR signalling | 0.0391 | 10 |
| TW 37 | BCL-2, BCL-XL | apoptosis regulation | 0.0195 | 5 |
| Embelin | XIAP | apoptosis regulation | 0.0391 | 10 |
| Erlotinib | EGFR | EGFR signalling | 0.0078 | 2 |
| AZ628 | BRAF | ERK MAPK | 0.0078 | 2 |
| | | signalling | | |
| AG-014699 | PARP1/2 | Genome integrity | 0.0195 | 5 |
| Gemcitibine | nucleoside analog | DNA replication | 0.0391 | 10 |
| GSK269962A | ROCK1/2 | cytoskeleton | 0.0195 | 5 |
| SB-505124 | TGFbetaR-I (ALK5) | other | 0.0391 | 10 |
| Tamoxifen | ER | other | 0.0195 | 5 |
| Fulvestrant | ER | other | 0.0039 | 1 |
| Anastrozole | ER | other | 0.0391 | 10 |
| JQ1 | BRD2, BRD3, BRD4 | chromatin other | 0.0039 | 1 |
| YK 4-279 | RNA helicase A | other | 0.0391 | 10 |
| CHIR-99021 | GSK3B | WNT signalling | 0.0391 | 10 |
| (5Z)-7-Oxozeaenol | TAK1 | other | 0.0391 | 10 |
| FK866 | NAMPT inhibitor | metabolism | 0.0039 | 1 |
| BMS-345541 | IKK-beta | other | 0.0391 | 10 |
| AZ960 | JAK2 | other | 0.0391 | 10 |
| BMN-673 | PARP | Genome integrity | 0.0391 | 10 |
| XAV 939 | Tankyrase (PARP5a) | WNT signalling | 0.0195 | 5 |
| GSK1120212 | MEK1, MEK2 | ERK MAPK | 0.0039 | 1 |
| | | signalling | | |
| GSK2118436 | BRAF | ERK MAPK | 0.0391 | 10 |
| | | signalling | | |
| Temozolomide | DNA akylating agent | DNA replication | 0.1172 | 30 |
| Olaparib + | DNA damage response | Genome integrity | 0.0391 | 10 |
| Temozolomide | | | | |
| AZD2281 | PARP | Genome integrity | 0.0391 | 10 |
| Bicalutamide | Androgen receptor | other | 0.0391 | 10 |
| PF-562271 | FAK | cytoskeleton | 0.0391 | 10 |
| PAC-1 | Caspase 3 activator | apoptosis regulation | 0.0391 | 10 |
| INCB-18424 | JAK1, JAK2, TYK2 | other | 0.0391 | 10 |
| OSI-906 | IGFR-1 | IGFR signalling | 0.0098 | 2.5 |
| Epirubicin | DNA damage | DNA replication | 0.0391 | 10 |
| Cyclophosphamide | DNA akylating agent | DNA replication | 0.0391 | 10 |
| Carboplatin | DNA damage | DNA replication | 0.0391 | 10 |
| Everolimus | mTOR | TOR signalling | 0.0195 | 5 |
| LCL161 | SMAC mimetic | apoptosis regulation | 0.0391 | 10 |
| rTRAIL | Death receptor ligand | apoptosis regulation | 0.39 ng/ml | 100 ng/ml |
| DMSO | CONTROL | NA | | |

TABLE 2
BAP1 mutation status in selected cell lines.

| | | | | | BAPI |
|---|---|---|---|---|---|
| | | | | rTRAIL | mRNA |
| SAMPLE_NAME | COSMIC_ID | DESCRIPTION | ZYGOSITY | response | expression |
| H226 | | Deletion | Homozygous | Sensitive | |
| H2461 | 1290810 | Deletion - | heterozygous | Sensitive | 0.32 |
| | | Frameshift | | | |
| H2722 | 1290812 | HomDel | homozygous | Resistant | −2.52 |
| H2731 | 1240134 | Essential Splice | heterozygous | Sensitive | −0.41 |
| H2795 | 1290813 | Essential Splice | heterozygous | Sensitive* | −0.45 |
| H2804 | 1240136 | Essential Splice | heterozygous | Sensitive | 0.51 |
| IST-MES1 | 907173 | Essential Splice | homozygous | Unknown | −0.33 |
| NCI-H2452 | 908462 | Substitution - | homozygous | Resistant | −0.04 |
| | | Missense | | | |
| NCI-H28 | 908470 | Essential Splice | heterozygous | Sensitive | 1.27 |
| NCI-H226 | 905941 | HomDel | homozygous | Sensitive** | ND |
| H2595 | 1240132 | Wild-type | heterozygous | Unknown | −2.05 |
| H2369 | 1290808 | Wild-type | heterozygous | Resistant | −0.64 |
| H2373 | 1290809 | Wild-type | heterozygous | Resistant | 1.65 |
| H2591 | 1240131 | Wild-type | heterozygous | Resistant | 0.93 |
| H2803 | 1240135 | Wild-type | heterozygous | Resistant | 0.62 |
| H2810 | 1240137 | Wild-type | heterozygous | Resistant | 1.06 |
| H2818 | 1290814 | Wild-type | heterozygous | Resistant | 0.41 |
| H2869 | 1240138 | Wild-type | heterozygous | Resistant | −0.67 |
| H290 | 1240139 | Wild-type | heterozygous | Unknown | −0.15 |
| H513 | 1240141 | Wild-type | heterozygous | Resistant | 0.11 |
| MPP-89 | 908150 | Wild-type | heterozygous | Resistant | 0.54 |
| MSTO-211H | 908152 | Wild-type | heterozygous | Sensitive** | −0.28 |
| NCI-H2052 | 688058 | Wild-type | heterozygous | Resistant | 0.92 |

### Example 2

### Modulation of BAP1 Expression Determines TRAIL Sensitivity Through Activation of Cell Death

TRAIL binds via two active transmembrane death receptors, DR4 and DR5, triggering a caspase cascade and subsequently cell death. The viability effect of TRAIL observed in BAP1 mutant cells was indeed associated with an increased fraction of cells stained with the apoptotic marker Annexin V (see FIG. 3B).

The inventors therefore next examined whether modulation of BAP1 expression in mesothelioma cells resulted in changes in TRAIL sensitivity. The ablation of BAP1 protein with the use of a lentiviral shRNA in the BAP1 wild-type cell line H2818 promoted a shift towards increased sensitivity in the BAP1 null compared to the BAP1 competent parental line (see FIG. 4). The BAP1 null cell line NCI-H226, which possesses a homozygous deletion of BAP1, was transduced with a BAP1 expression vector to restore expression of wild-type full length BAP1 (see FIG. 5A) or the catalytically dead C91A mutant. Treatment of the null NCI-H226 cell line with a dose range of TRAIL resulted in increased cell death which was significantly reduced in the BAP1 expressing H226 cell line (see FIG. 5b). The C91A variant however phenocopied the response of the BAP1 null parental cell line indicating a functional Ubiquitin hydrolase catalytic domain is critical for sensitivity to TRAIL. The nuclear localization signal (NLS) also plays a key role in imparting TRAIL resistance as deletion of NLS results in significant reduction in BAP1 induced TRAIL resistance (see FIG. 5c).

### Example 3

### Loss of BAP1 Expression and Function Modulates Components of the Apoptotic Machinery

The H226 mesothelioma cell line harbours a homozygous deletion of BAP1, resulting in complete loss of BAP1 expression. The inventors further examined the effect of this catalytically inactive BAP1 on differential mRNA gene expression as well as carrying out a signalling pathway impact analysis (SPIA), as previously described (PMID 18990722). Among those pathways significantly altered when comparing wild-type versus c91a mt BAP1, was that of cell death pathways (see FIG. 6A) (http://www.genome.jp/dbget-bin/www.bgat?path:map04210). In particular, there was decreased expression of members of the IAP family in H226 cells stably transduced with the catalytically dead C91A mutant (see FIG. 6B). The largest effects were seen in CIAP2, and this was confirmed by western blot (see FIG. 6C).

### Example 4

### Combination Drug Screen Demonstrates Synergy Between SMAC Mimetic LCL161 and rTRAIL in BAP1 Competent Cell Lines

rTRAIL was used as an anchor drug in combination with the library of 94 single agent compounds described above. Synergy was described using delta AUC metric (ref Wessles et al) and this was correlated with the previously described genomic subgroups. The inventors have shown that drugs such as SMAC mimetic LCL161, DNA helicase inhibitor YK-4279 and the tyrosine kinase inhibitor sorafenib to increase the efficacy of DRL—induced apoptosis in otherwise resistant cells. One of the most synergistic findings of this screen was the association of sensitivity to the SMAC mimetic LCL161 and rTRAIL in BARI wild-type MPM (see FIG. 7A). This was validated by treating TRAIL resistant wild-type BAP1 expressing cells with combination of LCL161 and TRAIL. Further validation was also performed in the H226 cell lines stably expressing wild-type BAP1 that was previously demonstrated to be resistant to TRAIL alone (see FIGS. 5 and 7B-D). The combination of the IAP inhibitor, LCL161, and TRAIL showed a synergistic increase in cell death in the both mutant and wild-type line indicating that DRL induced cell death in BAP1 mutant and wild-type cells can be enhanced by combining with other agents (see FIG. 7B-D). FIG. 7D in particular shows that both BAP1 mutant and wild-type cells undergo cell death in response to treatment with the combination of TRAIL and LCL161. Endogenous SMAC/Diablo is a specific natural inhibitor of IAP's (14). This data suggests that in the BAP1 competent state, BAP1 loss can be phenocopied by specifically mimicking this inhibitory effect on IAPs resulting in a net inactivation of IAP's and sensitivity to rTRAIL. This would lend further support to the idea that the BAP1/extrinsic apoptotic pathway perturbation seen is related to a specific dysregulation of net activity of IAP's.

### Example 5

### Extension of BAP1/TRAIL Effect to Other Tissues Harboring BAP1 Loss of Function Mutations

The deubiquitinase BAP1 is frequently mutated in pleural mesothelioma (36%), uveal melanoma (47%) and intrahepatic cholangiocarcinomas (25%) as previously noted. To determine whether additional BAP1 mutant tumours occur that may also be amenable to this therapeutic approach, the inventors extended this analysis to a cohort of 5180 tumour samples in 20 cancer types using variant data from The Cancer Genome Atlas (TOGA) (http://cancergenome.nih.gov/). Truncating BAP1 mutations were also observed in a diverse range of cancer types, with frequencies of up to 6% (see FIGS. 1b and c) Carbone, M. et al, Nature Reviews Cancer 13, 153-159 (March 2013). When the inventors extended their analysis to a panel of toot cancer cell lines that had previously been submitted for whole exome and copy number analysis, they identified 17 cell lines harbouring truncating mutations in BAP1 (http://cancer.sanger.ac.uk/cancergenome/projects/cell_lines/). These included clear cell kidney cancer, bladder cancer and breast cancer cell lines. Treatment of these cell lines with TRAIL resulted in a marked viability effect compared to BAP1 wild-type cell lines from the same cancer type (see FIG. 8A). The inventors also inactivated BAP1 using a lentiviral shRNA in the breast cancer cell line, MDA-MB231, and observed an exaggerated apoptotic response to rTRAIL (see FIG. 8B). This suggests that TRAIL therapy may be efficacious in other forms of cancer (in addition to mesothelioma), and that inhibition of BAP1 or by targeting the pathway which BAP1 induces TRAIL resistance it is possible to sensitise cancer cells to TRAIL.

The inventors demonstrated the efficacy of targeting BAP1 mutant cells with TRAIL in vivo by mice xenograft models. Mutant and wild-type BAP1 cells were injected subcutaneously into left and right flanks of mice. The mice were treated with either rTRAIL or vehicle (see FIG. 9A). The tumours were weighed at the end of the experiment and the weights of mutated BAP1 tumours in mice that received TRAIL was significantly less than wild-type BAP1 tumours with TRAIL treatment or mutant and wild-type tumours of mice with vehicle treatment (see FIG. 9B). The tumour burden was tracked in real time over a period of 4 weeks. The tumour burden of mutated BAP1 xenografts in mice that received TRAIL was significantly less than wild-type BAP1 xenografts with TRAIL treatment or mutant and wild-type xenografts of mice with vehicle treatment (see FIG. 9C).

### Example 6

### Role of ASXL Binding Site on of BAP1 Function

The inventors have also demonstrated that a mutation in the ASXL protein binding site of the BAP1 gene impairs BAP1-induced TRAIL resistance (see FIGS. 1A). BAP1 has been shown to form a complex with proteins ASXL1, ASXL2 or ASXL3. Mutation of the binding site for ASXL protein inhibits formation of BAP1-ASXL complexes. The BAP1-ASXL complex has been shown to deubiquitinate Histone 2A, and other substrates, and both BAP1 and ASXL1, ASXL2, or ASXL3 are required for this deubiquitination function. This complex is an important regulator of the Polycomb Respressor Complex and gene transcription. The inventors have shown that the BAP1 wild-type and ASXL3 mutant (truncating mutation) cell line H513 is TRAIL sensitive. Hence loss of function of ASXL1, ASXL2 or ASXL3 increases the sensitivity of cells to DRL induced cell death. Mutations of ASXL1, ASXL2 or ASXL3 also predict sensitivity to DLR and hence can be used as a biomarker for cell death independent of BAP1 mutational status.

### Example 7

### Extension of BAP1/TRAIL Effect to Other Extrinsic Death Pathways

Although the data in this application focus on rTRAIL, a recombinant protein that activates the TRAIL pathway by binding to DR4 and DR5 receptors, the observed BAP1 mutation-sensitisation extends to other extrinsic apoptotic pathways including the FAS ligand pathway and the TNFalpha pathway (see FIG. 10).

### SUMMARY

The inventors have found that BAP1 is an important regulator of whether a cell will undergo cell death in response to the activation of a death receptor by a death receptor ligand, such as TRAIL, TNF alpha (TNFα) and FAS ligand (FASL). Specifically, non-functional or low expression of wild-type BAP1 causes cells to become sensitive to death receptor ligand-induced cell death. Consequently, it has been discovered that a mutant BAP1 gene or a mutant BAP1 protein, or a cancer cell with low expression of a wild-type BAP1 protein may be used as a biomarker of sensitivity to DRL-induced cell death.

Thus, the invention also encompasses an advantageous:
method of determining if an individual's cancer cell is sensitive to death receptor ligand (DRL)-induced cell death;
kit for determining if an individual's cancer cell is sensitive to DRL-induced cell death;
method of selectively inducing death receptor ligand induced cell death in an individual suffering from a cancer that is insensitive to death receptor ligand induced cell death;
a method of sensitising to DRL-induced cell death, an individual suffering from a cancer that is insensitive to DRL-induced cell death;
a composition comprising a BAP1 inhibitor and a death receptor ligand; and
a method of treating, preventing or ameliorating an individual suffering from a cancer, which is insensitive to DRL-induced cell death.

### REFERENCES

1. Lanphear BP, Buncher CR. Latent period for malignant mesothelioma of occupational origin. Journal of occupational medicine : official publication of the Industrial Medical Association. 1992; 34(7)718-21. Epub Jul. 1, 1992.

2. Lemen RA, Dement JM, Wagoner JK. Epidemiology of asbestos-related diseases. Environmental health perspectives. 1980; 34:1-11. Epub Feb. 1, 1980.

3. Algranti E, Saito CA, Carneiro AP, Moreira B, Mendonca EM, Bussacos MA. The next mesothelioma wave: Mortality trends and forecast to 2030 in Brazil. Cancer epidemiology. 2015. Epub Sep. 1, 2015.

4. Treasure T, Waller D, Tan C, Entwisle J, O'Brien M, O'Byrne K, et al. The Mesothelioma and Radical surgery randomized controlled trial: the Mars feasibility study. Journal of thoracic oncology : official publication of the International Association for the Study of Lung Cancer. 2009; 4(10):1254-8. Epub Aug. 8, 2009.

5. Vogelzang NJ, Rusthoven JJ, Symanowski J, Denham C, Kaukel E, Ruffie P, et al. Phase III study of pemetrexed in combination with cisplatin versus cisplatin alone in patients with malignant pleural mesothelioma. Journal of clinical oncology : official journal of the American Society of Clinical Oncology. 2003; 21(14):2636-44. Epub Jul. _b 16, 2003.

6. Guo G, Chmielecki J, Goparaju C, Heguy A, Dolgalev I, Carbone M, et al. Whole-exome sequencing reveals frequent genetic alterations in BAP1, NF2, CDKN2A, and CULL in malignant pleural mesothelioma. Cancer research. 2015; 75(2):264-9. Epub Dec. 10, 2014.

7. Bott M, Brevet M, Taylor BS, Shimizu S, Ito T, Wang L, et al. The nuclear deubiquitinase BAP1 is commonly inactivated by somatic mutations and 3p21.1 losses in malignant pleural mesothelioma. Nature genetics. 2011; 43(7):668-72. Epub Jun. 7, 2011.

8. Martin M, Masshofer L, Temming P, Rahmann S, Metz C, Bornfeld N, et al.

Exome sequencing identifies recurrent somatic mutations in EIF1AX and SF3B1 in uveal melanoma with disomy 3. Nature genetics. 2013; 45(8):933-6. Epub Jun. 26, 2013.

9. Gerlinger M, Horswell S, Larkin J, Rowan AJ, Salm MP, Varela I, et al. Genomic architecture and evolution of clear cell renal cell carcinomas defined by multiregion sequencing. Nature genetics. 2014; 46(3)225-33. Epub Feb. 2, 2014.

10. Chan-On W, Nairismagi ML, Ong CK, Lim WK, Dima S, Pairojkul C, et al. Exome sequencing identifies distinct mutational patterns in liver fluke-related and non-infection-related bile duct cancers. Nature genetics. 2013; 45(12):1474-8. Epub Nov. 5, 2013.

11. Narasimhan SR, Yang L, Gerwin BI, Broaddus VC. Resistance of pleural mesothelioma cell lines to apoptosis: relation to expression of Bcl-2 and Bax. The American journal of physiology. 1998; 275(1 Pt 1):L165-71. Epub Aug. 5, 1998.

12. Hinds MG, Norton RS, Vaux DL, Day CL. Solution structure of a baculoviral inhibitor of apoptosis (IAP) repeat. Nature structural biology. 1999; 6(7):648-51. Epub Jul. 15, 1999.

13. de Almagro MC, Vucic D. The inhibitor of apoptosis (IAP) proteins are critical regulators of signaling pathways and targets for anti-cancer therapy. Experimental oncology. 2012; 34(3):200-11. Epub Oct. 17, 2012.

14. Yu J, Wang P, Ming L, Wood MA, Zhang L. SMAC/Diablo mediates the proapoptotic function of PUMA by regulating PUMA-induced mitochondrial events. Oncogene. 2007; 26(29):4189-98. Epub Jan. 24, 2007.

15. Behjati S et al. Recurrent PTPRB and PLCG1 mutations in angiosarcoma. Nat Genet, 2014 April; 46(4)376-9. doi: 10.1038/ng.2921.

16. Tarpey PS et al Frequent mutation of the major cartilage collagen gene COL2A1 in Chondrosarcoma. Nat Genet. 2013 August: 45(8): 923-926.

17. Li, H and Durbin, R Fast and accurate short read alignment with Burrows-Wheeler transform. Bioinformatics, 2009. 25(14): p1754-60.

18. Garnett, M.J., et al., Systemic identification of genomic markers of drug sensitivity in cancer cells. Nature, 2012. 483 (7391): p.473-81.

19. Behjati, S. et al., Mobile DNA in cancer. Extensive transduction of nonrepetitive DNA mediated by L1 retrotransposition in cancer genome. Science, 2014. 345(6196): p.1251343.

20. Ganten T, Koschny R, Sykora J, Schulze-Bergkamen H, Buchler P, Haas T, et al. Preclinical differentiation between apparently safe and potentially hepatotoxic applications of TRAIL either alone or in combination with chemotherapeutic drugs. Clin Cancer Res. 2006; 12:2640-6

1. A method of treating, preventing or ameliorating cancer in subject, the method comprising administering, to a subject in need thereof, a composition comprising (i) a BAP1 inhibitor or an agent that mimics the effect of BAP1 inhibition and (ii) a death receptor ligand.

2. The method according to claim 1, wherein the cancer is mesothelioma, Malignant Pleural Mesothelioma, uveal melanoma, melanoma, non-melanoma skin cancer, renal cancer, lung cancer, cancer of the pleura, abdominal cancer, peritoneal cancer, cancer of the pericardium, a head or neck cancer, brain cancer, breast cancer, liver or biliary tract cancer, gastrointestinal cancer including upper and lower tracts, urothelial cancer, prostate cancer, testicular cancer, cancer of the tunica vaginalis, ovarian cancer, cervical cancer, sarcoma, lymphoma or leukaemia.

3. The method according to claim 1, wherein the death receptor ligand is selected from a group consisting of: TRAIL; TNFalpha; FAS ligand (FASL); recombinant TRAIL (dulanermin); antibody to a death receptor; mapatumuab; drozitumumab; conatumumab; lexatumumab; tigatuzumab; Medi-3038; Medi-3039; and LBY-135; or a combination thereof.

4. The method according to claim 1, wherein the BAP1 inhibitor or agent that mimics the effect of BAP1 inhibition is selected from a group consisting of: an RNAi molecule; shRNA; siRNA; miRNA; ribozyme; antisense molecule; a TALEN (Transcriptional Activator Like-Effector Nuclease); a CRISPR/CAS9 nuclease; an IAP inhibitor; a SMAC mimetic; an inhibitor of BIRC2, BIRC3, BIRC5, BIRC6, BIRC7, BIRC8, NAIP or XIAP; LCL161; an RNA helicase inhibitor; YR-4279; a tyrosine kinase inhibitor; Sorafenib; WP1130, Usp9x, Usp5, Usp14, Usp24, UCH37, b-AP15, and a small molecule inhibitor of BAP1.

5. The method according to claim 2, wherein the cancer which is treated is mesothelioma.

6. The method according to claim 2, wherein the cancer which is treated is an asbestos-induced cancer.

7. The method according to claim 1, wherein the cancer which is treated is a cholangiocarcinoma.


SEQUENCE LISTING (44 sequences)

SEQ ID NO: 1 - DNA, 9102 bp, Homo sapiens
  source 1..9102: mol_type = unassigned DNA
gagcgcatgcccgcatctgctgtccgacaggcggaagacgagcccagaggcggagcagggccgtcgcgccttggtgacgtctgccgccggcgcgggcgggtgacgcgactgggcccgttgtctgtgtgtgggactgaggggccccgggggcggtgggggctcccggtgggggcagcggtggggagggagggcctggacatggcgctgaggggccgccccgcgggaagatgaataagggctggctggagctggagagcgacccaggtgaggaggggaccgggagggccaggggctggggaggccggatgggcccgggacgcgcctgcctgaccatcaccccctcctcttgtcgccccacccaggcctcttcaccctgctcgtggaagatttcggtaagagccttttctccctgccggaccggggctgtggcggcccacccctgcgccctcactcatcaggggctgtccttccctactgctttcctttcctcatcgcaggtgtcaagggggtgcaagtggaggagatctacgaccttcagagcaaatgtcaggggtgagtggctgtacaccagggctgccccttacacccagagtgctggggaaggtcccagagaacagggccccttagggaagacagtgccaggaaccctacgttgtaaaatctcacagaaagcagcagccttgctctctgagtgcccgctcctgatcaaactgatactttcttttctcccaaactttccttagcgcttccctttttgtagcagccccctccccacccctaagcatcctttggttcagctgctttcctggccttgcagcgggaagaccccggtcacacaatgtcttttgtgcagttgtgtaatgtattaattttagtgtgcccatgtgtccttggctttaatcctgacacaaagtcatcctgtattgattggttggggtgacaaggcccctcctgggtgcccacacttagagtcttttcccagtggtcctgcagaatagatgtgtaagagagtagcaacagtagcaaccgtgactgaaccaagaagtctactttaatttcctggaacaaaagagactggtgtgggtgttcatttgctttcctgactgcattggggcccacaagtgagaaggagtgcctcagttcctcatcagagtttttgttcttgtcttactttgtgttcctaccctgtcccatccttggccctcagttccagcttttcttctcttacccagaactatagacttcataaggagactgggtggactcctggagcatcacagtcagaggcttatgctttgctctgcctgtggcaggcctttggtgtgtgagggcacaaggccacttcagacacagtgttgggaagaagccaggggagaggggggatcacagcaaggacacctgagtgatgacgcagtgcaaaggattaatgggagaaagaagggaatgctgattgtcttctcccctttggctgatctggctctgccccttacttcccccagccctgtatatggatttatcttcctgttcaaatggatcgaagagcgccggtcccggcgaaaggtctctaccttggtggatgatacgtccgtgattgatgatgatattgtgaataacatgttctttgcccaccaggtctgctggactctgtgctttgtttggagggtgggatgctgccatgtttttgcttgggaagtggaaatggaggaagacaggaggaggagataggcagattctaggggtggtagctacagaaatcctctggcagaacgaactgaactcttaattcattaaagggaacagctttagagtaggagggtgtctgagtccactctctgtgtcctcagatatccagtgggtatttggtaggtgcttgttaaatgaataaacattaggcaaagatgaaaggagctgagaaggggagttgtccagatatgactgacctgctctggatccccattcttgatgtatatgggcttggggcttgcagtgaggggtgctgtgtatgggtgactattcttggtttcacagctgatacccaactcttgtgcaactcatgccttgctgagcgtgctcctgaactgcagcagcgtggacctgggacccaccctgagtcgcatgaaggacttcaccaagggtttcagccctgaggtaggctgcagtgccttcatcctggctcacagccaactgggcagatctgaccctgagggccactgggaatgctaccacatgatattgggtactattaggctgtttctttttcaaatgattgtttatgttacatttgactcttaaataaattgtgtaaggccattgtttttagatgcagttgcggggaaaggacacaggcctagggagggaggagagtttccttaagtcagaccatgtcagaaccttctctgtcaggacttttcctctcaggccatgttgcttcctagtgtccactaattaccatgcaaggccagcacagtccatctctttggggctccagagctcttttctgcccccaccagccttttaagaaagttcgtctgtgttccttccgattcctggaatgcctccaggctgctctctgaagctttgccttccacccatagtcctacctgaggagaaattattctgatacggccttattttcttccccgtagagcaaaggatatgcgattggcaatgccccggagttggccaaggcccataatagccatgccaggtgtgtgggagctgtgggagctgatgtggggtgggagtagggggagtatcattttttgggccctgactctgtttttccccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatggtaggcaccatgagctggaggcctgttgggtgtctctgcctacctcctagggagctggggctcagggccctctggtatgtggtacccagtggcaggggttgtcggtaccgacacccggctctggctggggtttcaccctacaccatattgcccgaccagctcctgattccctggctcaactgctcttctctgtcttccttcccactcctggcctgcccaaactcagggtttccttctcgctgattccttgtcttggtctccactagggccctggggggaggacgaggagtggacagacaaggcccggcgggtcatcatggagcgtatcggcctcgccactgcagggtaagggccctgtgcctgccctgttctactctctggagctgtacctactttgggagggacagagagtatccaggtgatttgtaaattgcaaggccatatggtgaatctggcaagatcaggcttagatcatgggttctcaacttgttgtcttatttcctgcctgggctgcctgtggcctgctcctgggtgggctgggggaggggcaggcctcagtggagccttaggcagcccaggtctgctggttcacttccagataggcccctcatacagcttgttggaaggtaccagctcaggtgcctggcatgtatggctagtcgctgcctgcctgttggggtggggcctatacctacagctgcaggtgtgactgcagggagccctgccaggatatctgcctcaacctgatggcggggccggggcgggagctgctctcacggctgcggctgtgactgcagggagccctaccacgacatccgcttcaacctgatggcagtggtgcccgaccgcaggatcaagtatgaggccaggctgcatgtgctgaaggtgaaccgtcagacagtactagaggctctgcagcaggtaggtgccctttcttcctggcctctgcccagcccaaccctccctgcattcctcctcccttcccccacagcatttgtctctgattcgtgaacatactctcttgtagatctgggcttcagctaaccacatcttttctttgcccccattgtgggaaaggtgggacttggagtggggagggagaatagcttctaaaaggaagtttgggtttgggtgttttatttccctgtgagtgaatgggtagagccaaggccattattcctttaggtcctcagcccttagctatttaaggtagaagcccgggtctaccctttctcctctgagccctggattctgttgttagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacaggtactggggggtttgggacctcttgtggacctcagagccacccgctaatgtctgacatgggaggcctaaacagggaaagtctttttctggggatgtccttgggcagtgttcttcccccgtcagaaggtagagggagagcagtccttccctaaagaaaggcacctgtaaagggccgctgttaccacaggcccctgggcccttctctgtaatgtacactccctttcttgttttctctagaggcggtttttttttttttttttttttttttttttcttcctgcttcttttttcccatctcattctttgccctgtctcattgcgggatcatgacttagagcttgctgactcccattgcaccagctggctgggctgttcttctctgggaagtgctggttcacagggccggggagactgtgagcttttcttggagatcctactggaggtcctgcctgtgttcttgccctgtctcagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggtaagctgggagcacccttgcaggattctctacttgattctcttgagaggctgcaacaggcaattttcccatgtggttccttggtgttcatccttggcatggctgggtcaagctgcctgggcctgggttgctaggttcctctgcctgatatgaaaaggcccccacaacagcaggagcttagggaggcagggagagctcctttgaatttaatctagttacgtggctgtgggattaaatgtttaggtcacgctccttggtacaacttcatgggttgggttttactggcaaaataaaggcatgtgtttcagggcactctgtttctcttaaaacccctccgtggggttctatccagtgtaagtgggtggcagcctccccacaagccaaggacaggccatggaacagctggaggggttccgctgactcagtctggaaaaccatgttggctttctctctggctgtgagtgtctaggctcagcctgggccgagcagcacttgtttgtaactgccctggtctttgtcccaggaggaagaagacctggcggcaggtgtgggccgcagccgagttccagtccgcccaccccagcagtactcagatgatgaggatgactatgaggatgacgaggaggatgacgtgcagaacaccaactctgcccttaggtcagcccagctttctaaggctaccaggttctaggtgcttcggatcccatcctgaatatctcagtctgtgtctgagaatgccctgcagcagataatgttgagcacctgcggagtttggggccctgggggaggctggcatgatggggctgaccccaggtccccaggaagtttttggtgggctggggggtaaggctgagcacgtaagcttatatcatgtcctattggaagtggccttttagccaggccttgaaggattggttggggcagggatggaggagatgtgggtggtggggaggcagctttgctggaacacagggcattggcaaaaggccaggagtgggatggctggaatagaggaagtgtcttttgaggacacttggctgcagctgtcagaacttgatgccaggcttagcatggctagttcaagttgcttggaccaagtataaggagttttagggtcagcccctggaggtcgggatgtatttaagccattctgggtactgctgggtatggtcacctggcccgttcccttgcttcacatcttctcgggccccacaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacaggtgggccttggactggctcactggccacttggtgcacccaggagggaggagggaagtggccaagtgaccacaaagtgtcctgcactctgatgattttcttgtgacctctcttcccagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggtgagcctccgttgtggttttctcctttaatcctggcagagggtaaggcctgagctcctcctgcccaggtgccaagttcttgattggaactttggtgtgaagattggtggctggagccatgtgccagaagactttctgggttgggtggtggcaggggccttgataggcatggactcgctgctcatccttgcctctagctgcctattgctcgtggggctttgttgctggcccgccccgatcagaggtgcaatgctgggttttggcaggagctgctggcactgctgaagtgtgtggaggctgagattgcaaactatgaggcgtgcctcaaggaggaggtagagaagaggaagaagttcaaggtgggtgatttctccagttgcctgatctggcctctcccgaggtccactggtggctgctctggcaagattggctccagtgctctcagtcttcttctctcctacagattgatgaccagagaaggacccacaactacgatgagttcatctgcacctttatctccatgctggctcaggaaggtgaggggatgcgctgctgtcttaactggaatgccctgctgagggccgtgtccttcagctcccctcccctggcctctcctgaggcttgagcagaccttggggcacagggagggccatgagagcctcagctcctggcctgaggcagccagcacctgctcaagggtctctacctcttcgcaggcatgctggccaacctagtggagcagaacatctccgtgcggcggcgccaaggggtcagcatcggccggctccacaagcagcggaagcctgaccggcggaaacgctctcgcccctacaaggccaagcgccagtgaggactgctggccctgactctgcagcccactcttgccgtgtggccctcaccagggtccttccctgccccacttccccttttcccagtattactgaatagtcccagctggagagtccaggccctgggaatgggaggaaccaggccacattccttccatcgtgccctgaggcctgacacggcagatcagccccatagtgctcaggaggcagcatctggagttggggcacagcgaggtactgcagcttcctccacagccggctgtggagcagcaggacctggcccttctgcctgggcagcagaatatatattttacctatcagagacatctatttttctgggctccaacccaacatgccaccatgttgacataagttcctacctgactatgctttctctcctaggagctgtcctggtgggcccaggtccttgtatcatgccacggtcccaactacagggtcctagctgggggcctgggtgggccctgggctctgggccctgctgctctagccccagccaccagcctgtccctgttgtaaggaagccaggtcttctctcttcattcctcttaggagagtgccaaactcagggacccagcactgggctgggttgggagtagggtgtcccagtggggttggggtgagcaggctgctgggatcccatggcctgagcagagcatgtgggaactgttcagtggcctgtgaactgtcttccttgttctagccaggctgttcaagactgctctccatagcaaggttctagggctcttcgccttcagtgttgtggccctagctatgggcctaaattgggctctaggtctctgtccctggcgcttgaggctcagaagagcctctgtccagcccctcagtattaccatgtctccctctcaggggtagcagagacagggttgcttataggaagctggcaccactcagctcttcctgctactccagtttcctcagcctctgcaaggcactcagggtgggggacagcaggatcaagacaacccgttggagcccctgtgttccagaggacctgatgccaaggggtaatgggcccagcagtgcctctggagcccaggccccaacacagccccatggcctctgccagatggctttgaaaaaggtgatccaagcaggcccctttatctgtacatagtgactgagtggggggtgctggcaagtgtggcagctgcctctgggctgagcacagcttgacccctctagcccctgtaaatactggatcaatgaatgaataaaactctcctaagaatctcctgagaaatgaa

SEQ ID NO: 2 - DNA, 2190 bp, Homo sapiens
  source 1..2190: mol_type = unassigned DNA
atgaataagggctggctggagctggagagcgacccaggcctcttcaccctgctcgtggaagatttcggtgtcaagggggtgcaagtggaggagatctacgaccttcagagcaaatgtcagggccctgtatatggatttatcttcctgttcaaatggatcgaagagcgccggtcccggcgaaaggtctctaccttggtggatgatacgtccgtgattgatgatgatattgtgaataacatgttctttgcccaccagctgatacccaactcttgtgcaactcatgccttgctgagcgtgctcctgaactgcagcagcgtggacctgggacccaccctgagtcgcatgaaggacttcaccaagggtttcagccctgagagcaaaggatatgcgattggcaatgccccggagttggccaaggcccataatagccatgccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatgggccctggggggaggacgaggagtggacagacaaggcccggcgggtcatcatggagcgtatcggcctcgccactgcaggggagccctaccacgacatccgcttcaacctgatggcagtggtgcccgaccgcaggatcaagtatgaggccaggctgcatgtgctgaaggtgaaccgtcagacagtactagaggctctgcagcagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggaggaagaagacctggcggcaggtgtgggccgcagccgagttccagtccgcccaccccagcagtactcagatgatgaggatgactatgaggatgacgaggaggatgacgtgcagaacaccaactctgcccttaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggagctgctggcactgctgaagtgtgtggaggctgagattgcaaactatgaggcgtgcctcaaggaggaggtagagaagaggaagaagttcaagattgatgaccagagaaggacccacaactacgatgagttcatctgcacctttatctccatgctggctcaggaaggcatgctggccaacctagtggagcagaacatctccgtgcggcggcgccaaggggtcagcatcggccggctccacaagcagcggaagcctgaccggcggaaacgctctcgcccctacaaggccaagcgccagtga

SEQ ID NO: 3 - protein, 729 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 4 - protein, 729 aa, Homo sapiens
MNKGWLELESDPGLFTLLVEDFGVKGVQVEEIYDLQSKCQGPVYGFIFLFKWIEERRSRQKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 5 - DNA, 2190 bp, Homo sapiens
  source 1..2190: mol_type = unassigned DNA
atgaataagggctggctggagctggagagcgacccaggcctcttcaccctgctcgtggaagatttcggtgtcaagggggtgcaagtggaggagatctacgaccttcagagcaaatgtcagggccctgtatatggatttatcttcctgttcaaatggatcgaagagcgccggtcccggcaaaaggtctctaccttggtggatgatacgtccgtgattgatgatgatattgtgaataacatgttctttgcccaccagctgatacccaactcttgtgcaactcatgccttgctgagcgtgctcctgaactgcagcagcgtggacctgggacccaccctgagtcgcatgaaggacttcaccaagggtttcagccctgagagcaaaggatatgcgattggcaatgccccggagttggccaaggcccataatagccatgccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatgggccctggggggaggacgaggagtggacagacaaggcccggcgggtcatcatggagcgtatcggcctcgccactgcaggggagccctaccacgacatccgcttcaacctgatggcagtggtgcccgaccgcaggatcaagtatgaggccaggctgcatgtgctgaaggtgaaccgtcagacagtactagaggctctgcagcagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggaggaagaagacctggcggcaggtgtgggccgcagccgagttccagtccgcccaccccagcagtactcagatgatgaggatgactatgaggatgacgaggaggatgacgtgcagaacaccaactctgcccttaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggagctgctggcactgctgaagtgtgtggaggctgagattgcaaactatgaggcgtgcctcaaggaggaggtagagaagaggaagaagttcaagattgatgaccagagaaggacccacaactacgatgagttcatctgcacctttatctccatgctggctcaggaaggcatgctggccaacctagtggagcagaacatctccgtgcggcggcgccaaggggtcagcatcggccggctccacaagcagcggaagcctgaccggcggaaacgctctcgcccctacaaggccaagcgccagtga

SEQ ID NO: 6 - DNA, 22 bp, synthetic construct
  misc_feature 1..22: note = Synthetic Primer
  source 1..22: mol_type = other DNA
acctagaacctggtagccttag

SEQ ID NO: 7 - DNA, 22 bp, synthetic construct
  misc_feature 1..22: note = Synthetic Primer
  source 1..22: mol_type = other DNA
gtacagctccagagagtagaac

SEQ ID NO: 8 - DNA, 22 bp, synthetic construct
  misc_feature 1..22: note = Synthetic Primer
  source 1..22: mol_type = other DNA
tcttaccgaaatcttccacgag

SEQ ID NO: 9 - DNA, 22 bp, synthetic construct
  misc_feature 1..22: note = Synthetic Primer
  source 1..22: mol_type = other DNA
ctgctgctttctgtgagatttt

SEQ ID NO: 10 - DNA, 21 bp, synthetic construct
  misc_feature 1..21: note = Synthetic Primer
  source 1..21: mol_type = other DNA
agggcattccagttaagacag

SEQ ID NO: 11 - DNA, 18 bp, synthetic construct
  misc_feature 1..18: note = Synthetic Primer
  source 1..18: mol_type = other DNA
caagagtgggctgcagag

SEQ ID NO: 12 - DNA, 21 bp, synthetic construct
  misc_feature 1..21: note = Synthetic Primer
  source 1..21: mol_type = other DNA
actaaggccattctgcttctc

SEQ ID NO: 13 - DNA, 20 bp, synthetic construct
  misc_feature 1..20: note = Synthetic Primer
  source 1..20: mol_type = other DNA
atcccaccctccaaacaaag

SEQ ID NO: 14 - DNA, 18 bp, synthetic construct
  misc_feature 1..18: note = Synthetic Primer
  source 1..18: mol_type = other DNA
caccaagtggccagtgag

SEQ ID NO: 15 - DNA, 20 bp, synthetic construct
  misc_feature 1..20: note = Synthetic Primer
  source 1..20: mol_type = other DNA
ggctgtcatcctctccaaaa

SEQ ID NO: 16 - DNA, 18 bp, synthetic construct
  misc_feature 1..18: note = Synthetic Primer
  source 1..18: mol_type = other DNA
gagggctgcgagtgtgtg

SEQ ID NO: 17 - DNA, 22 bp, synthetic construct
  misc_feature 1..22: note = Synthetic Primer
  source 1..22: mol_type = other DNA
ctctgccaggattaaaggagaa

SEQ ID NO: 18 - DNA, 18 bp, synthetic construct
  misc_feature 1..18: note = Synthetic Primer
  source 1..18: mol_type = other DNA
gaatgcagggagggttgg

SEQ ID NO: 19 - DNA, 22 bp, synthetic construct
  misc_feature 1..22: note = Synthetic Primer
  source 1..22: mol_type = other DNA
acccaatatcatgtggtagcat

SEQ ID NO: 20 - DNA, 18 bp, synthetic construct
  misc_feature 1..18: note = Synthetic Primer
  source 1..18: mol_type = other DNA
aaggacagcccctgatga

SEQ ID NO: 21 - DNA, 19 bp, synthetic construct
  misc_feature 1..19: note = Synthetic Primer
  source 1..19: mol_type = other DNA
gtaggcagagacacccaac

SEQ ID NO: 22 - DNA, 22 bp, synthetic construct
  misc_feature 1..22: note = Synthetic Primer
  source 1..22: mol_type = other DNA
ccttctctggtcatcaatctgt

SEQ ID NO: 23 - DNA, 20 bp, synthetic construct
  misc_feature 1..20: note = Synthetic Primer
  source 1..20: mol_type = other DNA
ctctgaggtccacaagaggt

SEQ ID NO: 24 - DNA, 22 bp, synthetic construct
  misc_feature 1..22: note = Synthetic Primer
  source 1..22: mol_type = other DNA
tcaagtagagaatcctgcaagg

SEQ ID NO: 25 - DNA, 22 bp, synthetic construct
  misc_feature 1..22: note = Synthetic Primer
  source 1..22: mol_type = other DNA
gagcagcacttgtttgtaactg

SEQ ID NO: 26 - DNA, 22 bp, synthetic construct
  misc_feature 1..22: note = Synthetic Primer
  source 1..22: mol_type = other DNA
ctcaactgctcttctctgtctt

SEQ ID NO: 27 - DNA, 18 bp, synthetic construct
  misc_feature 1..18: note = Synthetic Primer
  source 1..18: mol_type = other DNA
gagggagggcctggacat

SEQ ID NO: 28 - DNA, 21 bp, synthetic construct
  misc_feature 1..21: note = Synthetic Primer
  source 1..21: mol_type = other DNA
ctgtccttccctactgctttc

SEQ ID NO: 29 - DNA, 22 bp, synthetic construct
  misc_feature 1..22: note = Synthetic Primer
  source 1..22: mol_type = other DNA
gaagttcaaggtgggtgatttc

SEQ ID NO: 30 - DNA, 18 bp, synthetic construct
  misc_feature 1..18: note = Synthetic Primer
  source 1..18: mol_type = other DNA
ctcagctcctggcctgag

SEQ ID NO: 31 - DNA, 23 bp, synthetic construct
  misc_feature 1..23: note = Synthetic Primer
  source 1..23: mol_type = other DNA
ggagaaattattctgatacggcc

SEQ ID NO: 32 - DNA, 22 bp, synthetic construct
  misc_feature 1..22: note = Synthetic Primer
  source 1..22: mol_type = other DNA
gaagggaatgctgattgtcttc

SEQ ID NO: 33 - DNA, 18 bp, synthetic construct
  misc_feature 1..18: note = Synthetic Primer
  source 1..18: mol_type = other DNA
ctatccgctcagccaacc

SEQ ID NO: 34 - DNA, 22 bp, synthetic construct
  misc_feature 1..22: note = Synthetic Primer
  source 1..22: mol_type = other DNA
ctctcaattcctctgtccatca

SEQ ID NO: 35 - DNA, 20 bp, synthetic construct
  misc_feature 1..20: note = Synthetic Primer
  source 1..20: mol_type = other DNA
cgttcccttgcttcacatct

SEQ ID NO: 36 - DNA, 22 bp, synthetic construct
  misc_feature 1..22: note = Synthetic Primer
  source 1..22: mol_type = other DNA
tcctgcactctgatgattttct

SEQ ID NO: 37 - DNA, 22 bp, synthetic construct
  misc_feature 1..22: note = Synthetic Primer
  source 1..22: mol_type = other DNA
gatatctgcctcaacctgatgg

SEQ ID NO: 38 - DNA, 20 bp, synthetic construct
  misc_feature 1..20: note = Synthetic Primer
  source 1..20: mol_type = other DNA
gtgctgtgtatgggtgacta

SEQ ID NO: 39 - DNA, 21 bp, synthetic construct
  misc_feature 1..21: note = Synthetic Primer
  source 1..21: mol_type = other DNA
gaagatgaataagggctggct

SEQ ID NO: 40 - DNA, 19 bp, synthetic construct
  misc_feature 1..19: note = Synthetic Primer
  source 1..19: mol_type = other DNA
tgatgtggggtgggagtag

SEQ ID NO: 41 - DNA, 18 bp, synthetic construct
  misc_feature 1..18: note = Synthetic Primer
  source 1..18: mol_type = other DNA
cccgatcagaggtgcaat

SEQ ID NO: 42 - DNA, 22 bp, synthetic construct
  misc_feature 1..22: note = Synthetic Primer
  source 1..22: mol_type = other DNA
agctatttaaggtagaagcccg

SEQ ID NO: 43 - DNA, 22 bp, synthetic construct
  misc_feature 1..22: note = Synthetic Primer
  source 1..22: mol_type = other DNA
actgtgagcttttcttggagat

SEQ ID NO: 44 - DNA, 19 bp, synthetic construct
  misc_feature 1..19: note = Synthetic sequence for short hairpin
  source 1..19: mol_type = other DNA
taaaggtgcagatgaactc